SEQUENCE LISTING (19 sequences)

SEQ ID NO: 1 - protein, 6 aa, Artificial Sequence
  Description of Artificial Sequence: Synthetic 6xHis tag
HHHHHH

SEQ ID NO: 2 - protein, 5 aa, Artificial Sequence
  Description of Artificial Sequence: Synthetic peptide
DDDDK

SEQ ID NO: 3 - protein, 6 aa, Artificial Sequence
  Description of Artificial Sequence: Synthetic peptide
LVPRGS

SEQ ID NO: 4 - protein, 8 aa, Artificial Sequence
  Description of Artificial Sequence: Synthetic peptide
LEVLFQGP

SEQ ID NO: 5 - protein, 7 aa, Artificial Sequence
  Description of Artificial Sequence: Synthetic peptide
ENLYFQS

SEQ ID NO: 6 - protein, 7 aa, Artificial Sequence
  Description of Artificial Sequence: Synthetic peptide
ENLYFQG

SEQ ID NO: 7 - protein, 6 aa, Artificial Sequence
  Description of Artificial Sequence: Synthetic peptide
LVPRGS

SEQ ID NO: 8 - protein, 13 aa, Artificial Sequence
  Description of Artificial Sequence: Synthetic peptide
GRRRYLEVLFQGP

SEQ ID NO: 9 - protein, 13 aa, Artificial Sequence
  Description of Artificial Sequence: Synthetic peptide
LEVLFQGPYRRRG

SEQ ID NO: 10 - protein, 11 aa, Artificial Sequence
  Description of Artificial Sequence: Synthetic peptide
ENLYFQGGGGS

SEQ ID NO: 11 - protein, 11 aa, Artificial Sequence
  Description of Artificial Sequence: Synthetic peptide
LVPRGSYRRRG

SEQ ID NO: 12 - protein, 6 aa, Artificial Sequence
  Description of Artificial Sequence: Synthetic peptide
LVPRGS

SEQ ID NO: 13 - protein, 13 aa, Artificial Sequence
  Description of Artificial Sequence: Synthetic peptide
LEVLFQGPGGGGS

SEQ ID NO: 14 - protein, 13 aa, Artificial Sequence
  Description of Artificial Sequence: Synthetic peptide
ENLYFQGGYRRRG

SEQ ID NO: 15 - protein, 13 aa, Artificial Sequence
  Description of Artificial Sequence: Synthetic peptide
GGGGSYENLYFQG

SEQ ID NO: 16 - protein, 5 aa, Artificial Sequence
  Description of Artificial Sequence: Synthetic peptide
GGGGS

SEQ ID NO: 17 - protein, 5 aa, Artificial Sequence
  Description of Artificial Sequence: Synthetic peptide
HHHHH

SEQ ID NO: 18 - protein, 10 aa, Artificial Sequence
  Description of Artificial Sequence: Synthetic peptide
  SITE (1)..(10): This sequence may encompass 7-10 residues
  misc_feature: See specification as filed for detailed description of substitutions and preferred embodiments
HHHHHHHHHH

SEQ ID NO: 19 - protein, 40 aa, Artificial Sequence
  Description of Artificial Sequence: Synthetic polypeptide
  SITE (1)..(40): This sequence may encompass 1-8 "Gly Gly Gly Gly Ser" repeating units
  misc_feature: See specification as filed for detailed description of substitutions and preferred embodiments
GGGGSGGGGSGGGGSGGGGSGGGGSGGGGSGGGGSGGGGS

(19) United States
(12) Patent Application Publication
(10) Pub. No.: US 2023/0176063 A1
(43) Pub. Date: Jun. 8, 2023

(54) PHOTOREACTIVE AND CLEAVABLE PROBES FOR TAGGING BIOMOLECULES

(71) Applicant: Academia Sinica, Taipei (TW)

(72) Inventors: Chih-Wei CHANG, Hsinchu (TW); Yi-De CHEN, Yilan County (TW); Jung-Chi LIAO, Taipei (TW)

(21) Appl. No.: 17/913,101

(22) PCT Filed: Mar. 19, 2021
(86) PCT No.: PCT/US2021/023282

§ 371 Date: Sep. 20, 2022

Related U.S. Application Data
(60) Provisional application No. 62/992,253, filed on Mar. 20, 2020.

(51) Int. Cl. G01N 33/58

(52) U.S. Cl. CPC G01N 33/581

(57) ABSTRACT

Compositions including photoreactive and cleavable probes and methods of using the probes. The probes may include a tag conjugatable to a label, a cleavable linker linkable to a bait molecule, and a light activated warhead. The compositions and methods may be useful for analyzing biomolecules.

### CROSS REFERENCE TO RELATED APPLICATIONS

This application claims priority to Provisional Patent Application No. 62/992,253 filed Mar. 20, 2020.

### SEQUENCE LISTING

The instant application contains a Sequence Listing which has been submitted electronically in ASCII format and is hereby incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and compositions for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples.

### BACKGROUND

Cells are composed of different types of biological molecules (biomolecules). The biomolecules in the cells interact with neighbor biomolecules in the subcellular environment to form complexes, organelles, or other assemblies and to carry out various essential cell functions. Characterizing the subcellular environment, within which biomolecules interact with one another, and how the biomolecules function together is very challenging. Biomolecules are small, and they exist in a cell environment with tens of millions of other molecules. The interactions between neighboring biomolecules are frequently weak, and techniques used to study biomolecules disrupt their interactions. While techniques such as yeast two-hybridization assays and more recently proximity labeling have advanced our understanding of the cell environment, these techniques suffer from various limitations such as nonspecific binding, slow reaction times and disruption of the natural cell environment, resulting in false positives and missed interactions. What is needed are better tools for determining naturally occurring biomolecule interactions. Described herein are systems, compositions, and methods to better analyze endogenous biomolecule interactions.

### SUMMARY OF THE DISCLOSURE

Described herein are systems, compositions, and methods to better analyze endogenous biomolecule interactions. The methods and compositions may be useful for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples. These probes may be especially useful for selectively tagging and proximity labeling of biomolecules via selective light illumination through a microscope system.

One aspect of the disclosure provides a photoreactive and cleavable probe. Some embodiments of the photoreactive and cleavable probe include a multivalent core including a plurality of attachment sites. Some embodiments include a tag bound to one of the attachment sites, wherein the tag is configured to conjugate to a label. Some embodiments include a cleavable linker bound to a second of the attachment sites and configured to link to a bait molecule, wherein the cleavable linker includes a cleavable linker bond other than a disulfide bond. Some embodiments include a light-activated warhead bound to a third of the attachment sites.

In some embodiments, the probe is bioorthogonally cleavable.

In some embodiments, the tag includes a biotin derivative, a CLIP-tag, a click chemistry tag, digoxigenin, a peptide tag, a HaloTag, or a SNAP-tag. In some embodiments, the biotin derivative includes the moiety of

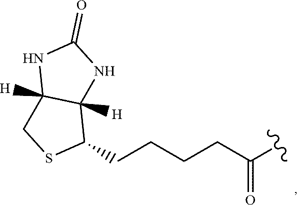

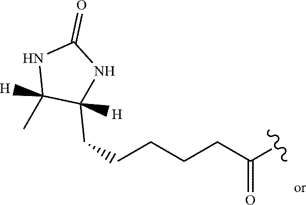

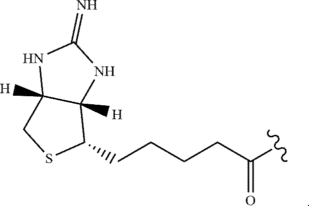

In some embodiments the click chemistry tag comprises an alkyne-based or azide-based moiety.

In some embodiments the click chemistry tag includes the moiety of

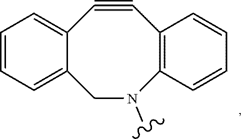

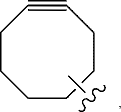

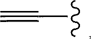

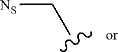

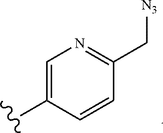

In some embodiments, the cleavable linker includes an azobenzene derivative, a boronic acid ester, a Dde derivative, a DNA oligomer, or a peptide.

In some embodiments, the azobenzene derivative includes the moiety of

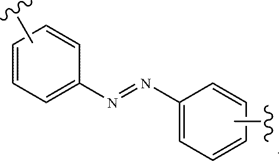

In some embodiments, the Dde derivative includes the moiety of

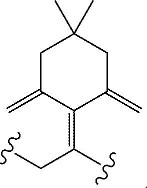

In some embodiments, the bait molecule includes an antibody, a CLIP-tag, a HaloTag, protein A, protein G, protein L, an RNA molecule, a small molecule, or a SNAP-tag.

In some embodiments, the light-activated warhead includes an aryl azide, a benzophenone, or a diazirine.

In some embodiments, the aryl azide comprises the moiety of

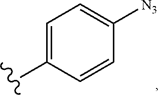

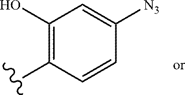

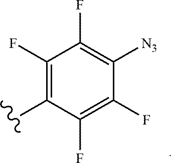

In some embodiments, the diazirine includes the moiety of

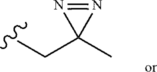

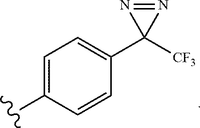

In some embodiments, the benzophenone includes the moiety of

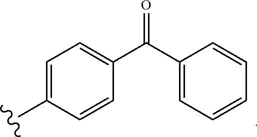

The photoreactive and cleavable probe of any of claims 1-10, wherein the light-activated warhead comprises a nucleobase-specific 3-cyanovinylcarbazole nucleoside (CNVK), including the moiety of

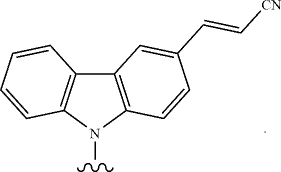

The photoreactive and cleavable probe of any of claims 1-10, wherein the light-activated warhead comprises a nucleobase-specific psoralen, including the moiety of

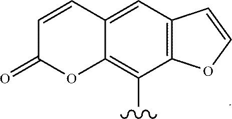

The photoreactive and cleavable probe any of claims 1-10, wherein the phenoxyl radical trapper comprises a light-activated warhead, including the moiety of

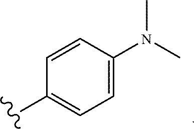

In some embodiments, the cleavable linker comprises azobenzene, boronic ester, a Dde moiety, a DNA oligomer, or a cleavable peptide.

In some embodiments, the cleavable linker comprises a human rhinovirus 3C (HRV 3C) protease recognition sequence or a tobacco etch virus (TEV) protease recognition sequence.

In some embodiments, the multivalent core includes the moiety of formula (I):

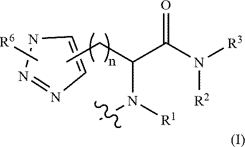

wherein n is 1, 2, 3, 4, 5, or 6;
R¹ and R² each independently are hydrogen, substituted
alkyl, substituted alkenyl, substituted alkynyl, substituted carbocyclyl, substituted heterocyclyl, substituted aryl, substituted heteroaryl, or a nitrogen protecting group; and
one of R³ and R⁴ is - (CH₂)ₓ(OCH₂CH₂)_{y}(CH2)_{z}NR⁵R⁶, and the other is an attachment site,
wherein x is 1, 2, 3, 4, 5, or 6;
y is 1, 2, 3, 4, 5, or 6;
z is 0, 1, 2, 3, 4, 5, or 6; and
one of R⁵ and R⁶ is an attachment site, and the other is
hydrogen, substituted alkyl, substituted alkenyl, substituted alkynyl, substituted carbocyclyl, substituted heterocyclyl, substituted aryl, substituted heteroaryl, or a nitrogen protecting group.

In some embodiments, the multivalent core comprises the moiety of formula (I-1) or (I-2):

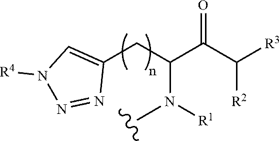

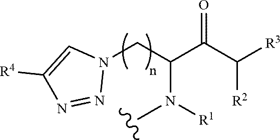

In some embodiments, the multivalent core comprises the moiety of:

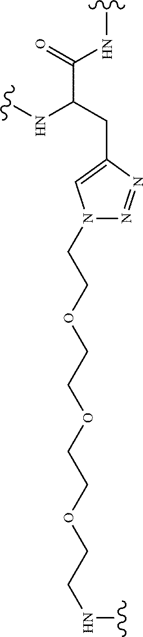

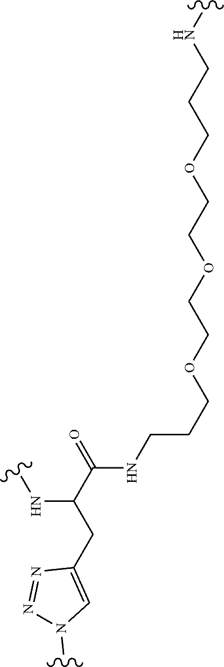

or

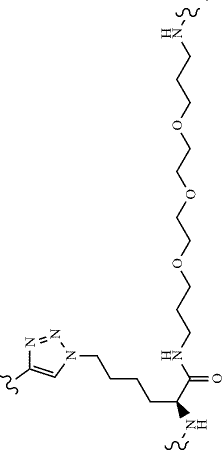

In some embodiments, the probe includes the following structure:

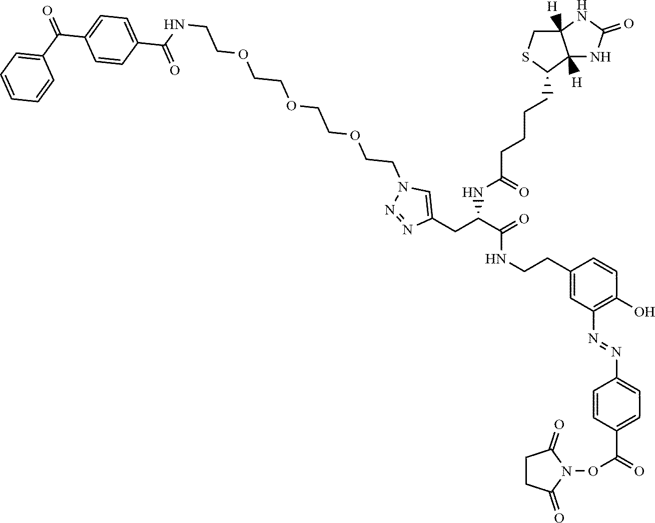 [SMILES: O=C(CCCC[C@@H]1SC[C@@H]2NC(=O)N[C@@H]21)N[C@@H](Cc1cn(CCOCCOCCOCCNC(=O)c2ccc(C(=O)c3ccccc3)cc2)nn1)C(=O)NCCc1ccc(O)c(/N=N/c2ccc(C(=O)ON3C(=O)CCC3=O)cc2)c1]

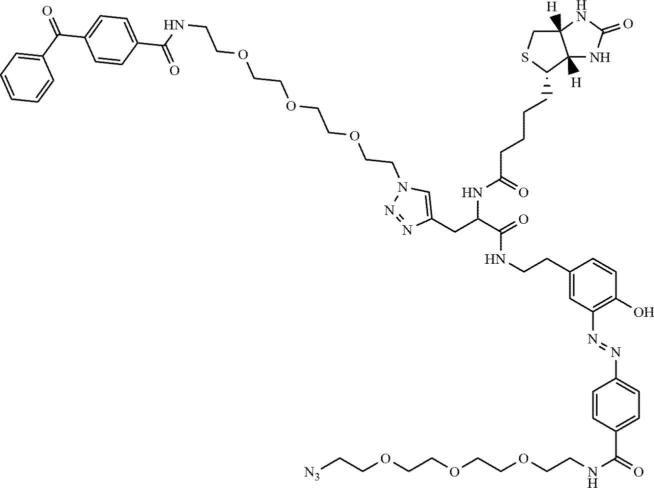 [SMILES: [N-]=[N+]=NCCOCCOCCOCCNC(=O)c1ccc(/N=N/c2cc(CCNC(=O)C(Cc3cn(CCOCCOCCOCCNC(=O)c4ccc(C(=O)c5ccccc5)cc4)nn3)NC(=O)CCCC[C@@H]3SC[C@@H]4NC(=O)N[C@@H]43)ccc2O)cc1]

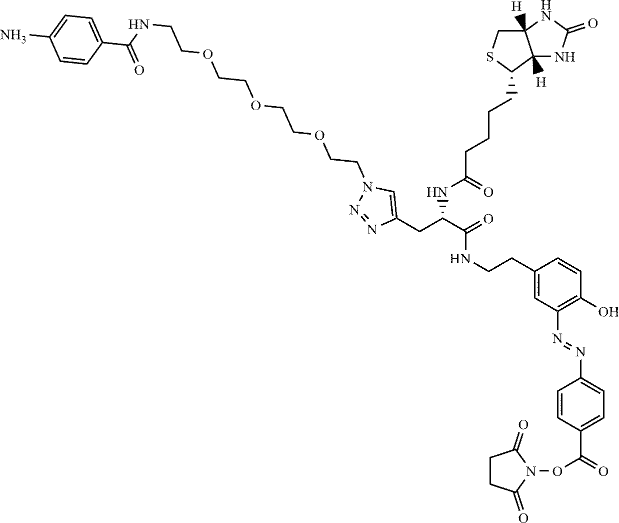 [SMILES: Nc1ccc(C(=O)NCCOCCOCCOCCn2cc(C[C@H](NC(=O)CCCC[C@@H]3SC[C@@H]4NC(=O)N[C@@H]43)C(=O)NCCc3ccc(O)c(/N=N/c4ccc(C(=O)ON5C(=O)CCC5=O)cc4)c3)nn2)cc1]

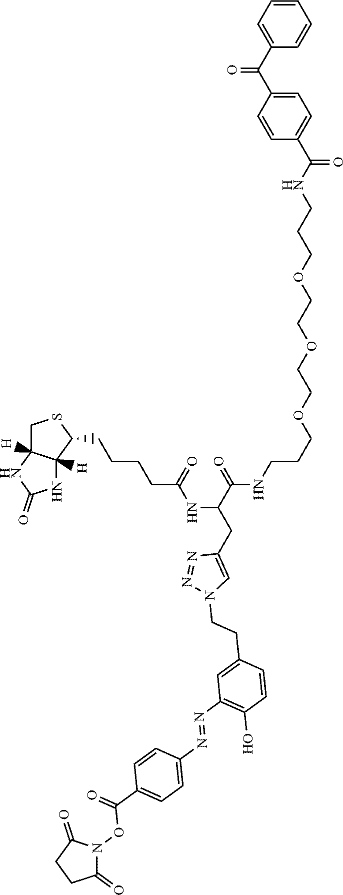 [SMILES: O=C(CCCC[C@H]1SC[C@H]2NC(=O)N[C@H]21)NC(Cc1cn(CCc2ccc(O)c(/N=N/c3ccc(C(=O)ON4C(=O)CCC4=O)cc3)c2)nn1)C(=O)NCCCOCCOCCOCCCNC(=O)c1ccc(C(=O)c2ccccc2)cc1]

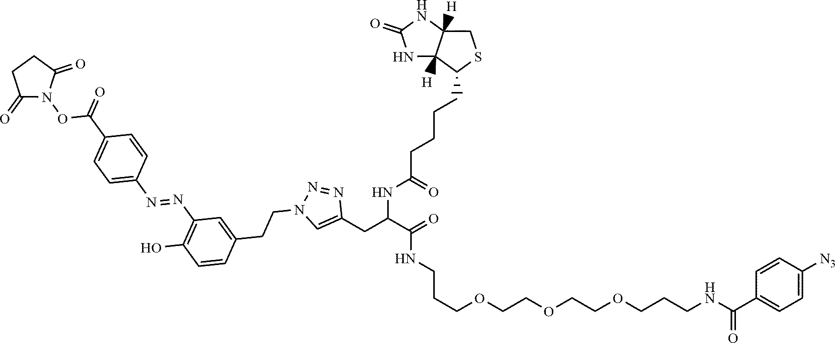 [SMILES: [N-]=[N+]=Nc1ccc(C(=O)NCCCOCCOCCOCCCNC(=O)C(Cc2cn(CCc3ccc(O)c(/N=N/c4ccc(C(=O)ON5C(=O)CCC5=O)cc4)c3)nn2)NC(=O)CCCC[C@H]2SC[C@H]3NC(=O)N[C@H]32)cc1]

or

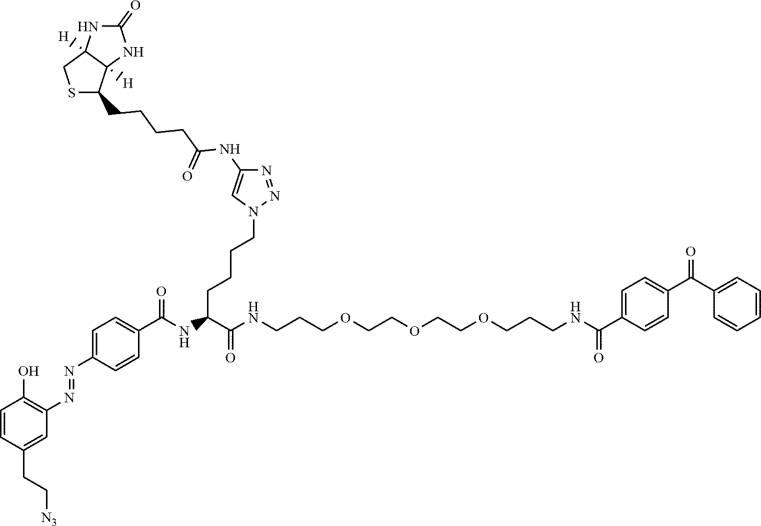 [SMILES: [N-]=[N+]=NCCc1ccc(O)c(/N=N/c2ccc(C(=O)N[C@@H](CCCCn3cc(NC(=O)CCCC[C@H]4SC[C@H]5NC(=O)N[C@H]54)nn3)C(=O)NCCCOCCOCCOCCCNC(=O)c3ccc(C(=O)c4ccccc4)cc3)cc2)c1]

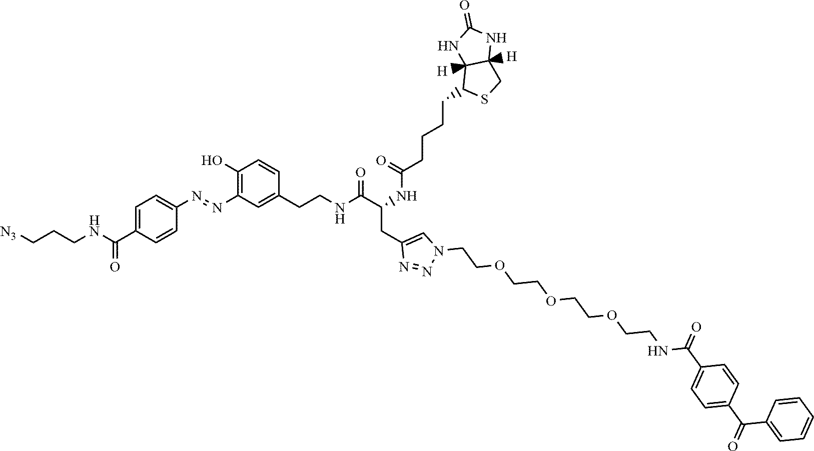

In some embodiments, the probes 2 and 6 further include an additional linker molecule configured for linking the probes 2 and 6, respectively, to the bait molecule.

Some embodiments further include a flexible linker. In some embodiments, the flexible linker includes polyethylene glycol (PEG) or an (GGGGS)n oligomer (SEQ ID NO: 16).

Another aspect of the disclosure provides a method delivering the photoreactive and cleavable probe as claimed in any one of claims above to a biological sample, wherein the photoreactive and cleavable probe is linked to the bait molecule; conjugating the bait molecule to a target biomolecule in the biological sample to crosslink the probe and target biomolecule; delivering optical radiation to activate the light-activated warhead of the photoreactive and cleavable probe and attach the warhead to the target biomolecule or a target biomolecule neighbor such that the probe and target biomolecule are double-crosslinked; cleaving the cleavable linker of the probe such that probe that is not double-crosslinked to the target biomolecule or a target biomolecule neighbor is cleaved; and removing the cleaved and unbound probe.

Another aspect of the disclosure provides a method of photoactivated tagging and proximity labeling including delivering a photoreactive and cleavable probe to a biological sample, wherein the probe comprises a cleavable linker, a light-activated warhead, and a tag and attached to a core of the probe; conjugating the bait molecule to a target biomolecule in the biological sample to crosslink the probe and target biomolecule; illuminating the biological sample from an imaging lighting source of an image-guided microscope system; imaging the illuminated sample with a controllable camera; acquiring with the camera at least one image of subcellular morphology of the biological sample in a first field of view; processing the at least one image and determining a region of interest in the sample based on the processed image; obtaining coordinate information of the region of interest; selectively illuminating the region of interest with optical radiation to activate the light-activated warhead and attach the warhead to the target biomolecule or a target biomolecule neighbor such that the probe and target molecule are double-crosslinked; cleaving the cleavable linker of the probe such that probe that is not double-crosslinked to the target biomolecule or a target biomolecule neighbor is cleaved; and removing the cleaved and unbound probe.

In some of these embodiments, the step of cleaving the cleavable linker includes performing a bioorthogonal cleavage reaction.

In some of these embodiments, the cleavable linker comprises a cleavable linker bond and the step of cleaving the cleavable linker comprises cleaving a bond other than a disulfide bond

Some embodiments further include the step of conjugating a detectable label with the tag of the probe and detectably labeling neighbors proximal the target biomolecule by detectable label activity.

In some embodiments, the step of detectable proximity labeling further includes photoselective proximity labeling a region less than 300 nm, less than 200 nm, or less than 100 nm in diameter.

In some embodiments of the method, the detectable label includes a catalytic label.

In some embodiments of the method, the biological sample includes a plurality of cells. In some embodiments of the method, the biological sample comprises at least one, at least 100, at least 1000 or at least 10,000 live or fixed cells. In some embodiments of the method, the biological sample includes fixed cells, tissues or cell or tissue extracts.

In some embodiments of the method, selectively illuminating includes illuminating a zone defined by point spread function.

In some embodiments of the method, wherein the biological sample is disposed on a microscope stage, the method further includes removing at least a portion of the region of interest from the stage.

In some embodiments of the method, the method further includes subjecting the sample to mass spectrometry analysis or sequencing analysis.

In some embodiments of the method, the tag includes a biotin derivative, a CLIP-tag, a click chemistry tag, digoxigenin, a HaloTag, a peptide tag, or a SNAP-tag.

In some embodiments of the method, the tag includes the click chemistry tag includes an alkyne-based or azide-based moiety.

In some embodiments of the method, the cleavable linker includes an azobenzene derivative, a boronic acid ester, a Dde derivative, a DNA oligomer, or a peptide.

In some embodiments of the method, the bait molecule comprises an antibody, a CLIP-tag, a HaloTag, protein A, protein G, protein L, a small molecule, or a SNAP-tag.

In some embodiments of the method, the light-activated warhead comprises an aryl azide, a benzophenone, or a diazirine.

Another aspect of the disclosure provides photoreactive and cleavable probe including a multivalent core having a plurality of attachment sites and a tag bound to one of the attachment sites, wherein the tag is configured to conjugate to a label. Some embodiments include a cleavable linker bound to a second of the attachment sites and configured to link to a bait molecule, wherein the cleavable linker includes a peptide sequence.

Some embodiments include a light-activated warhead bound to a third of the attachment sites, wherein the multivalent core comprises the moiety of formula (II) or (III):

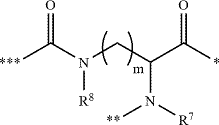

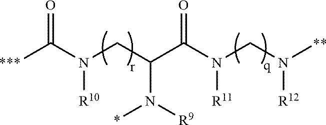

wherein m, r and q each independently are 1, 2, 3, 4, 5, or 6;
wherein * includes an attachment site of one of the plurality of attachment sites for the cleavable linker, wherein ** includes a different attachment site of the plurality of attachment sites for one of either the tag or the photoreactive warhead; wherein *** includes a different attachment site of the plurality of attachment sites for either the photoreactive warhead or the tag, respectively, and R7, R8, R9, R10, R11, and R12 each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In some embodiments, ** includes the attachment site for the tag, and *** includes the attachment site for the photoreactive warhead.

In some embodiments, the peptide sequence includes a protease recognition sequence. In some embodiments, the peptide sequence includes a human rhinovirus 3C (HRV 3C) protease recognition sequence, a tobacco etch virus (TEV) protease recognition sequence, or a thrombin recognition sequence.

In some embodiments, the cleavable linker further includes a conjugatable amino acid configured to conjugate to a bait molecule.

In some embodiments, the cleavable linker further includes a cysteine or clickable amino acid. In some embodiments, the cleavable linker includes a clickable amino acid with an azido or alkyne moiety. Another aspect of the invention provides a kit for labeling biomolecules including the photoreactive and cleavable probe of any of claims above in a first container; and an instructional material.

Another aspect of the invention provides a kit for labeling biomolecules includes a multivalent core moiety in a first container, wherein the multivalent core includes a plurality of attachment sites; and an instructional material.

In some embodiments, the kit includes a tag configured to conjugate to a label and bound to or configured to bind to the multivalent core moiety.

In some embodiments, the kit includes a cleavable linker having a cleavable linker bond other than a disulfide bond, wherein the cleavable linker is linked to or configured to link to a bait molecule.

In some embodiments, the kit includes a light-activated warhead bound to or configured to bind to a third attachment site on the multivalent core moiety.

In some embodiments of the kit, the multivalent core, the tag, the cleavable linker, and the light-activated warhead are present in the same molecule.

In some embodiments of the kit, at least one of the tag, the cleavable linker, and the light-activated warhead are separate from the multivalent core.

Some embodiments of the kit further include a linker cleavage molecule. In some embodiments of the kit, the linker cleavage molecule includes an endonuclease or a site-specific protease. In some embodiments of the kit, the linker cleavage molecule includes human rhinovirus 3C (HRV 3C) protease or tobacco etch virus (TEV) protease. In some embodiments of the kit, the linker cleavage molecule includes factor X enteropeptidase or thrombin.

Some embodiments of the kit further include one or more of an antioxidant, a buffering agent, a detergent, a nuclease inhibitor, a stabilizing agent, and a wash agent.

Some embodiments of the kit further include a bait molecule.

Some embodiments of the kit further include a detectable label.

Some embodiments of the kit further include a label that specifically conjugates with biotin.

Some embodiments of the kit further include a fixative solution.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a schematic depiction of a system useful for photoselective spatial tagging and proximity labeling of cells on a substrate.

FIG. 2A shows a schematic illustration of a multifunctional photoreactive and cleavable probe. The photoreactive and cleavable probe has a multivalent core with a plurality of attachment sites. A tag, a cleavable linker, and a light activated warhead are bound to the attachment sites on the probe. FIG. 2B schematically illustrates a proximity labeling system that can be used to label biomolecules in a small region of interest using the probe shown in FIG. 2A.

FIG. 2C shows a schematic illustration comparing the results of direct photochemical labeling with photo-assisted enzymatic proximity labeling using the multifunctional photoreactive and cleavable probes described herein to label biomolecules in small region of interest (ROI). The probes are shown in FIG. 2B.

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A). With mild cleavage conditions the protein structure is retained, and the reaction is bioorthogonal, while with harsh cleavage conditions, the protein denatures, and the reaction is non-bioorthogonal.

FIGS. 4A-4K show examples of tags that can be used in the photoreactive and cleavable probes described herein. The tags are configured to interact with a label for labeling biomolecules neighboring a target molecule of interest. FIG. 4A-FIG. 4E show examples of click chemistry tags that can be used with the probes. FIGS. 4F-4H show examples of biotin derivatives that can be used with the probes. FIG. 4I shows a digoxigenin moiety. FIG. 4J shows a peptide tag (SEQ ID NO: 1). FIG. 4K shows a SNAP-tag.

FIGS. 5A-5E show examples of site-specific cleavable linkers that can be used in the photoreactive and cleavable probes described herein. FIG. 5A shows an azobenzene moiety. FIG. 5B shows a boronic ester moiety. FIG. 5C shows a Dde moiety. FIG. 5D shows a DNA oligomer. FIG. 5E shows a peptide moiety.

FIGS. 6A-6E shows examples of bait molecules that can be used in the photoreactive and cleavable probes described herein to conjugate with a molecule of interest in a sample. FIG. 6A shows an antibody that can be used a bait molecule. FIG. 6B shows a nucleic acid portion that can be used as a bait molecule. FIG. 6C shows a representation of a functional protein that can be used as a bait molecule. FIG. 6D shows small molecules/drugs can be used as bait molecules. By way of example, erlotinib is shown. FIG. 6E shows a CLIP-tag and other members of self-labeling moieties could be used (e.g., HaloTag or SNAP-Tag).

FIGS. 7A-7I show examples of photoactive warheads that can be used in the photoreactive and cleavable probes described herein.

FIGS. 8A-8G show additional examples of linkers that can be used in the photoreactive and cleavable probe described herein.

FIGS. 9A-9G show examples of photoreactive and cleavable probes. The probes have multivalent cores with a plurality of attachment sites. A tag is bound to one of attachment sites, a cleavable linker is bound to another attachment site, and a light activated warhead is bound to another of the attachment sites on the probe.

FIGS. 10A-10B schematically illustrate peptide-based photoreactive and cleavable probes. These probes have a peptide region cleavable by a peptide cleavage reagent, such as by a protease that recognizes a specific peptide sequence. FIG. 10A shows an example of a peptide-based probe with a tag and warhead on the N-terminal end of the peptide region. FIG. 10B shows an example of a peptide-based probe with a tag and warhead on the C-terminal end of the peptide region. FIGS. 10A-10B also show probes with an additional, flexible linker and an optional clickable amino acid. Additional linkers (also referred to as spacers) can play a role in bridging the attachment sites between bait molecules and photoreactive and cleavable probe. The distance between the probe and bait can be controlled by applying linkers with different spatial lengths.

FIGS. 10C-10I show examples of reactive or clickable amino acids that can be used with the probes shown in FIGS. 10A and 10B. A clickable amino acid may be useful for attaching a bait molecule, such as an antibody.

FIGS. 10J-10Q show examples of peptide-based photoreactive and cleavable probes schematically illustrated in FIGS. 10A-10B. The cleavage sites for the human rhinovirus 3C (HRV 3C) protease, tobacco etch virus (TEV) protease, and thrombin are shown with arrows. FIGS. 10J-10Q disclose SEQ ID NOS 8-15, respectively, in order of appearance.

FIGS. 11A-11D illustrate methods and steps used to synthesize the photoreactive and cleavable probes described herein. The methods create probes with a tag, a cleavable linker, and a light activated warhead. FIG. 11D discloses SEQ ID NOS 10 and 10.

FIG. 12A schematically illustrates a photoreactive and cleavable probe conjugated to an antibody bait.

FIG. 12B and FIG. 12C schematically illustrates a reaction scheme for performing photoselective tagging of a molecule using a photoreactive and cleavable probe conjugated to an antibody bait for tagging proteins in the cell nucleolus. FIG. 12B illustrates how the reaction proceeds using controlled light. FIG. 12B illustrates how the cleavable probes are cleaved to reduce background in non-lighted areas.

FIG. 12D shows results from using the reaction schemes shown in FIG. 12A and FIG. 12B. The nucleolin protein is specifically tagged in the presence of light (top and right panels) but is not tagged in the absence of light (bottom panel).

FIG. 13A represents a schematic diagram of an imaging-guided system.

FIG. 13B depicts the optical path of the image-guided system of FIG. 13A.

FIG. 14A represents a schematic diagram of another imaging-guided system.

FIG. 14B depicts the optical path of the image-guided system of FIG. 14A.

FIG. 15A represents a schematic diagram of yet another imaging-guided system.

FIG. 15B depicts the optical path of the image-guided system of FIG. 15A.

### DETAILED DESCRIPTION

Described herein are systems, compositions, and methods useful for identifying, tagging, obtaining, and analyzing biomolecules and their neighboring biomolecules. The compositions and methods may be particularly useful for analyzing biomolecule interactions in biological samples, such as analyzing proteins, nucleic acids, carbohydrates, or lipids in cell or tissue samples. The compositions and methods utilize photoreactive and cleavable probes (e.g., bioorthogonally or mildly cleavable or enzyme-specific cleavage) that can label biomolecules and their neighboring biomolecules, while largely maintaining naturally occurring molecular structure in the biomolecules. The photoreactive and mildly cleavable probes described herein may be particularly useful for specifically labeling subsets of biomolecules in subcellular regions of cells using an image guided microscope with precision illumination control such as the system described in U.S. Pat. Publication No. 2018/0367717, to enable automatic labeling of cellular biomolecules of interest. The probes can be used for in situ tagging of biomolecules such as proteins inside cells or tissues and that can be followed by tag transfer or proximity labeling such as using Tyramide Signal Amplification (TSA). The biomolecules can be further analyzed by analytical techniques such as mass spectrometry and sequencing. These probes may be especially useful for performing omics studies, such as genomics, proteomics, and transcriptomics, and for finding relevant biomarkers for diagnosis and treatment.

### Abbreviations and Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Amino acids described herein may be conservatively substituted so long as conservatively substituted peptide enables the desired function (such as recognition by a protease). Examples of conservative substitutions include Thr, Gly, or Asn for Ser and His, Lys, Glu, Gln for Arg. Conservative substitutions are described in e.g., Molecular Cloning: A Laboratory Manual, Fourth Edition, Green and Sambrook, eds., Cold Spring Harbor Laboratory Press, Cold Spring Harbor 2014, as well as corrections and updates thereto.)

The term “antibody” refers to immunoglobulin and related molecules and includes monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also includes antibody fragments. An antibody may be a polyclonal or monoclonal or recombinant antibody. Antibodies may be murine, human, humanized, chimeric, or derived from other species. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope.

The term “aryl” refers to an aromatic ring system having a single ring (e.g., a phenyl group or a substituted phenyl group). Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

The term “bait molecule” refers to a molecule that specifically interacts with a molecule of interest, which may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “binding” refers to a first moiety physically interacting with a second moiety, wherein the first and second moieties are in physical contact with one another.

The term “bioorthogonal” refers to not interfering with or not interacting with biology (e.g., being inert to biomolecules).

The term “bioorthogonal reaction” or “bioorthogonal cleavage reaction” refers to a reaction that proceeds under physiologically relevant conditions and compatibility with naturally occurring functional groups and typically with fast kinetics, tolerance to an aqueous environment, and high selectively. A bioorthogonal reaction proceeds under conditions configured to maintain naturally occurring molecular structure, such as protein folding or three-dimensional structure. A bioorthogonal reaction does not break cross-links between different regions of polypeptide chains in endogenous or sample proteins or peptides. For example, a bioorthogonal reaction does not break covalent bonds in naturally occurring functional groups (e.g., disulfide (—S—S— bonds) in cysteine side chains). A bioorthogonal cleavage linker or a cleavage linker in a bioorthogonal cleavage probe is configured for bioorthogonal cleavage, such as being compatible with using an enzyme or bond-specific chemicals configured to proceed bioorthogonally without breaking covalent bonds in naturally occurring functional groups.

The term “biotin derivative” refers to a biotin moiety, including biotin and variations of biotin, such as biotin with an open ring or substitutions. Typically, a biotin derivative is easily detectable with a biotin-binding entity or protein, such as avidin, NeutrAvidin, or streptavidin.

The term “catalyzed reporter deposition” (CARD) refers an enzyme catalyzed deposition of a detectable molecule on or near target biomolecules (e.g., carbohydrates, lipids, nucleic acids, or proteins). In some embodiments, the enzyme in an enzyme catalyzed deposition is horseradish peroxidase (HRP) and the detectable molecule is tyramide or digoxygenin (DIG).

The term “cleavable linker bond” refers to the chemical bond in a cleavable linker configured to be specifically cleaved by a cleavage reagent. Typically, a cleavable linker bond refers to a single bond; however, in some variations, a cleavable linker bond can refer to more than one bond, such as in the case of a double-stranded DNA cleavable linker cleavable by an endonuclease in which two strands of DNA are cleaved.

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper(I)-catalyzed [3+2]- Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797.) Other examples of click chemistry are nucleophilic substitutions; additions to C-C multiple bonds (e.g., Michael addition, epoxidation, dihydroxylation, aziridination); and nonaldol like chemistry (e.g., N-hydroxysuccinimide active ester couplings). Click chemistry reactions can be bioorthogonal reactions, but do not need to be.

The term “conjugate” refers to a process by which two or more molecules specifically interact. In some embodiments, a tag and a label conjugate. In some embodiments, a bait and a cleavable probe conjugate.

The term “conjugatable” refers to a molecule that can specifically come together with another molecule to which it can be conjugated. In some embodiments, a bait is conjugatable to a biomolecule of interest. In some embodiments a cleavable probe is conjugatable to a label.

The term “detectable label” refers to a compound or composition which is or is configured to be conjugated directly or indirectly to a molecule. The label itself may be detectable and be a directly detectable label (such as, e.g., fluorescent labels such as fluorescent chemical adducts, radioisotope labels, etc.), or the label can be indirectly detectable (such as, e.g., in the case of an enzymatic detectable label, the enzyme may catalyze a chemical alteration of a substrate compound or composition and the product of the reaction is detectable). Examples of detectable labels include e.g., a biotin label, a fluorescent label, horseradish peroxidase, an immunologically detectable label (e.g., a hemagglutinin (HA) tag, a poly-histidine tag), another light emitting label, and a radioactive label. An example of an indirect label is biotin, which can be detected using a streptavidin detection method.

The term “enzymatic cleavage reaction” refers to cleavage or hydrolysis of bonds in molecules mediated by an enzyme. Typically, enzyme mediated reactions cleave covalent bonds and lead to the formation of smaller molecules.

The term “immunoglobulin-binding protein” refers to immunoglobulin-binding bacterial proteins and variations of immunoglobulin-binding bacterial proteins. Examples include protein A, protein G, protein L, protein A/G, and protein A/G/L. Protein A and protein G and are bacterial proteins originally obtained from Staphylococcus aureus and Group G Streptococci, respectively, and have high affinity for the Fc region of IgG type antibodies. Protein A/G combines the binding domains of protein A and protein G. Protein A/G/L combines binding domains of protein A, protein G, and protein L. Immunoglobulin-binding proteins bind to specific domain of antibodies.

The term “instructional material” includes a publication, a recording, a diagram, a link, or any other medium of expression which can be used to communicate the usefulness of one or more compositions of the invention for its designated use. The instructional material of a kit of the invention may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The term “label” refers to a molecule which produces or can be induced to produce a detectable signal. In some embodiments, a label produces a signal for detecting a neighboring biomolecule. Examples of labels that can be used include avidin labels, NeutrAvidin labels, streptavidin labels to detect a biotin tag.

The term “linker” refers to a structure which connects two or more substructures. A linker has at least one uninterrupted chain of atoms extending between the substructures. The atoms of a linker are connected by chemical bonds, typically covalent bonds.

The term “light activated warhead” refers to a group with a light activated moiety. Examples of light activated warheads include aryl azides, benzophenone, and diazirines. Once activated, a light activated warhead can bind to a binding partner.

The term “mass spectrometer” refers to an instrument for measuring the mass-to-charge ratio of one or more molecules in a sample. A mass spectrometer typically includes an ion source and a mass analyzer. Examples of mass spectrometers includes matrix assisted laser desorption ionization (MALDI), continuous or pulsed electrospray (ES) ionization, ionspray, magnetic sector, thermospray, time-of-flight, and massive cluster impact mass spectrometry.

The term “mass spectrometry” refers to the use of a mass spectrometer to detect gas phase ions.

The term “mass spectrometry analysis” includes linear time-of-flight (TOF), reflectron time-of-flight, single quadruple, multiple quadruple, single magnetic sector, multiple magnetic sector, Fourier transform, ion cyclotron resonance (ICR) or ion trap.

The term “photoactivated” or “light activated” refers to excitation of atoms by means of radiant energy (e.g., by a specific wavelength or wavelength range of light, UV light, etc.). In some examples, a photoactivated molecule forms a covalent linkage with another molecule or another part of itself within its immediate vicinity.

The term “peptide” refers to a polymer in which the monomers are amino acids and the monomers are joined together through amide bonds. A peptide is typically at least 2, least 5, least 10, least 20, least 50, least 100, or at least 500 or more amino acids long.

The term “photoreactive group” refers to a functional moiety, which, upon exposure to light (e.g., a specific wavelength or wavelength range of light, UV light, etc.) becomes activated. A photoreactive group typically forms a covalent linkage with a molecule within its immediate vicinity.

The term “proximity molecule” or neighboring molecule refers to a molecule that is near another molecule. A proximity molecule or neighbor molecule may bound to the molecule (e.g., covalently or non-covalently) or may be close by and not bound to the molecule.

The term “prey” refers to a binding partner of a bait molecule. For example, if an antibody is a bait, a corresponding protein to which the bait molecule can bind is the corresponding prey. In some embodiments, a bait can bind with a single prey. In some embodiments, a bait can bind with more than one prey.

The term “protein tag” refers to peptide sequences of amino acids. Protein tags can typically be conjugated to a label. An example of a protein tag is a “self-labeling” tag. Examples of self-labeling tags include BL-Tag, CLIP-tag, covalent TMP tag, HALO-tag, and SNAP-tag. SNAP-tag is a ~20 kDa variant of the DNA repair protein O6-alkylguanine-DNA alkyltransferase that specifically recognizes and rapidly reacts with benzylguanine (BG) derivatives. During a labeling reaction, the benzyl moiety is covalently attached to the SNAP-tag, releasing guanine. CLIP-tag is a variation of SNAP-tag configured to react specifically with O2-benzylcytosine (BC) derivatives rather than benzylguanine (BG).

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than about 1000 molecular weight or less than about 500 molecular weight.

The term “tag” refers to a functional group, compound, molecule, substituent, or the like, that can enable detection of a target molecule. A tag can enable a detectable biological or physiochemical signal that allows detection via any means, e.g., absorbance, chemiluminescence, colorimetry, fluorescence, luminescence, magnetic resonance, phosphorescence, radioactivity. The detectable signal provided due to the tag can be directly detectable due to a biochemical or physiochemical property of the tag moiety (e.g., a fluorophore tag) or indirectly due to the tag interaction with another compound or agent. Typically, a tag is a small functional group or small organic compound. In some embodiments, the employed tag has a molecular weight of less than about 1,000 Da, 750 Da, 500 Da or even smaller.

The term “tagging” refers to the process of adding a tag to a functional group, compound, molecule, substituent, or the like. Typically, tagging enables detection of a target molecule.

The term “tyramide signal amplification” (TSA), refers to a catalyzed reporter deposition (CARD) an enzyme-mediated detection method that utilizes catalytic activity of an enzyme (e.g., horseradish peroxidase) to catalyze inactive tyramide to highly active tyramide. The amplification can take place in the presence of low concentrations of hydrogen peroxide (H₂O₂). In some examples, tyramide can be labeled with a detectable label, such as fluorophore (such as biotin or 2,4-dinitrophenol (DNP)).

The practice of the techniques described herein may employ, unless otherwise indicated, conventional techniques and descriptions of chemistry, biochemistry, cell biology, immunology, molecular biology (including cell culture, recombinant techniques, sequencing techniques) and organic chemistry technology which are explained in the literature in the field (e.g., Molecular Cloning: A Laboratory Manual, Fourth Edition, Green and Sambrook, eds., Cold Spring Harbor Laboratory Press, Cold Spring Harbor 2014, as well as corrections and updates thereto; John D. Roberts and Marjorie C. Caserio (1977) Basic Principles of Organic Chemistry, second edition. W. A. Benjamin, Inc., Menlo Park, CA.).

### Compositions

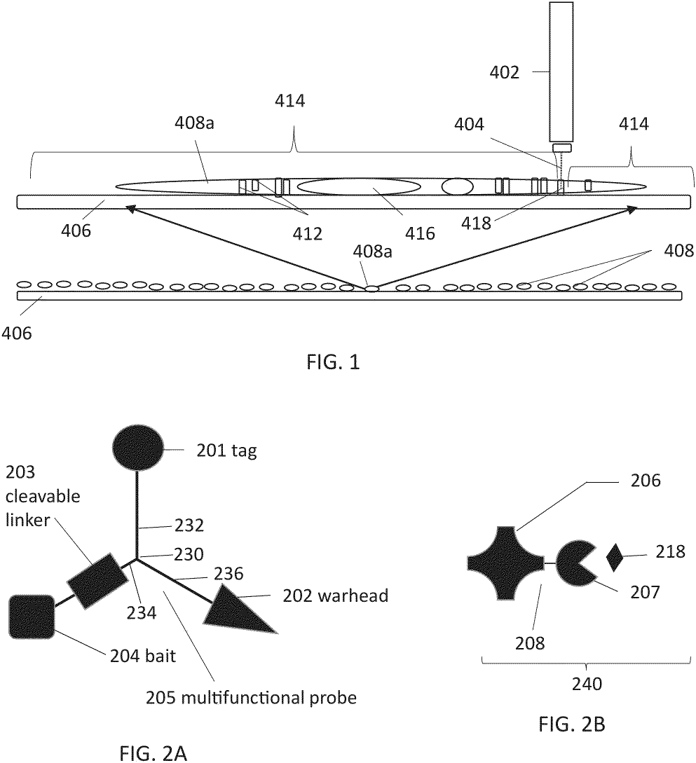

Described herein are compositions of matter including photoreactive and cleavable probes (e.g., bioorthogonally or mildly cleavable probes). The photoreactive and cleavable probes can advantageously be used with a microscope system, such as the systems described herein and in U.S. Pat. Publication No. 2018/0367717 A1, to enable automatic labeling of cellular biomolecules proximal to a biomolecule of interest. The labeled molecules may be adjacent the biomolecule of interest or may be close-by but not adjacent, such as when intervening molecules are between the biomolecule of interest and cellular biomolecules for capture or analysis. Molecules that are close-by but not adjacent to a molecule of interest may be part of cell structure or otherwise contribute to a cell microenvironment of interest. FIG. 1 shows a schematic depiction of a system useful for photoselective spatial tagging and labeling. The bottom part of FIG. 1 shows substrate 406, such as a microscope stage, and a monolayer of plurality of cells 408 disposed on the substrate. In some embodiments, the surface of an entire substrate, or a portion of the substrate, can be analyzed using an automated microscope system to identify a region of interest. For example, a sample can be stained or labeled to identify a region of interest. The top part of FIG. 1 shows an expanded view of cell 408a, one of the plurality of cells 408. The cell 408a has a nucleus 416 and a plurality of different types of organelles 412, such as cell membranes, mitochondria, ribosomes, and vacuoles. Microscope system 402 selectively shines narrow band of light 404 onto region of interest (ROI) 418 for analysis of the region of interest 418. The illumination can be selective, and large regions 414 of the cell and substrate are not illuminated. As explained in more detail below, narrow band of light 404 activates a photoreactive and mildly cleavable probe in only the region of interest 418.

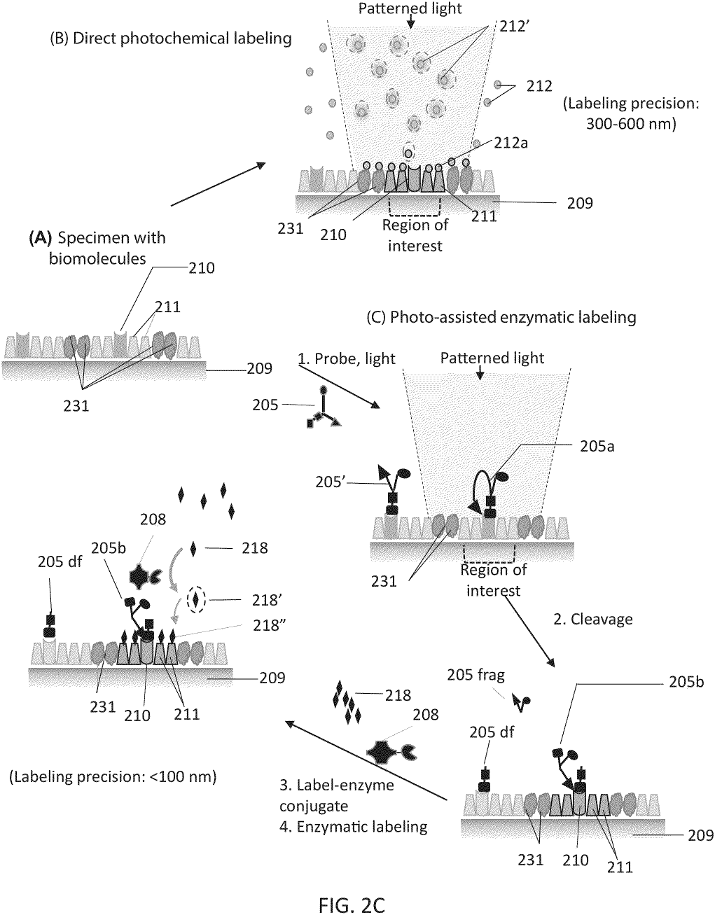

FIG. 2A schematically illustrates multifunctional probe 205 (also referred to interchangeably herein as multifunctional photoreactive and cleavable probe, photoreactive and cleavable probe, or probe unless specific context indicates otherwise). FIG. 2A shows multifunctional probe 205 has a multivalent core 230 with a plurality of attachment sites, first attachment site 232, second attachment site 234, and third attachment site 236. The multifunctional probe of FIG. 2A has tag 201 (circle) attached to first attachment site 232, cleavable linker 203 (rectangle) attached to second attachment site 234, and a light activated warhead (triangle) attached to third attachment site 236, thus forming a trivalent and trifunctional probe. Bait molecule 204 (rounded square) is attached to cleavable linker 203. FIG. 2B shows an example of a labeling system 240 that can be used with the multifunctional probe 205 shown in FIG. 2B to label biomolecules neighboring a target biomolecule of interest. Labeling system 240 includes labeling complex 208 with label 206 and enzyme or catalyst 207, and enzyme/catalyst substrate 218. In some embodiments, label 206 is NeutrAvidin and enzyme or catalyst 207 is peroxidase and utilizes peroxide (not shown) for activity. In this example, tag 201 and label 206 recognize one another and conjugate. Enzyme or catalyst 207 activates enzyme/catalyst substrate 218 and, once activated, activated enzyme/catalyst substrate 218 can bind to and detectably label biomolecules in its vicinity.

FIG. 2C shows a schematic illustration comparing the results of direct photochemical labeling with photo-assisted enzymatic labeling using the multifunctional photoreactive and cleavable probes described herein to label biomolecules in small region of interest (ROI).

FIG. 2B shows a comparison of direct photochemical labeling (top, labeled Process B) and photo-assisted enzymatic labeling (bottom, labeled Process C) using the probes and systems described herein on a specimen with biomolecules (A). Prior to performing either Process B or Process C, a sample (e.g., a cell or tissue sample) containing a biomolecule of interest 210 (protein will be used herein by way of example, but other biomolecules could instead be analyzed) is analyzed and a region of interest identified. The sample can be pretreated, such as fixed and stained. For example, a sample can be fixed and stained with a cell stain (e.g., hemotoxylin and eosin (H&E); Masson’s trichrome stain), identified with an immunofluorescent labeled antibody recognizing a protein of interest or by other methods. Once the region of interest is identified, a complex of neighboring biomolecules within the region of interest is analyzed. As illustrated in Process B, the sample is treated with a direct photoreactive probe 212 and patterned light is directed to the sample and activate direct photoreactive probe 212 to form activated direct photoreactive probe 212′. The activated direct photoreactive probe 212′ is able to form complexes with other molecules with a close vicinity (show by the dotted circle in Process B. The activated direct photoreactive probe 212′ can diffuse and labels neighbor molecules 211 near the molecule of interest 210. However, the labeling diameter (300-600 nm) of direct photoactivation of photoreactive probes is spatially restricted by the diffraction limit of the light sources used. Additionally, since the photoreactive probe is free to diffuse, any proteins in the pathway of the patterned light can be labeled. Process B also shows it labels more distant biomolecules 231. The region labeled by activated direct photoreactive probe 212′, or labeled precision, covers a region of about 300-600 nm. This region can include biomolecules that are not in close proximity to protein of interest, and in some cases might lead to confusing, misleading or unhelpful results.

In contrast, in Process C, shown on the bottom of FIG. 2C, multifunctional probe 205 preconjugated with bait molecule (see FIG. 2A) recognizing the biomolecule of interest is delivered to the sample on substrate 209. As illustrated in Step 1, patterned light is also directed to the sample. However, here, patterned light activates the photoreactive warhead 202 which binds to molecules or moieties close by. In addition to the light directing a limited region of activation, the photoreactive warhead is constrained by its attachment to the probe 205 and the photoreactive warhead becomes attached to the biomolecule of interest. The attached probe 205a is now double-crosslinked to the biomolecule of interest (or close to it). FIG. 2C also shows Step 2 Cleavage, and the cleavable linker 203 is cleaved, such as by the addition of a protease if the cleavable linked is a cleavable peptide linker. Step 1 and Step 2 also show how background or unwanted labeling is reduced using the probes and methods described herein. In Step 1, a probe 205′ is attached to a biomolecule; however, since the probe 205′ is outside the light delivery region, photoreactive warhead 202 is not activated and does not bind to the biomolecule of interest. In Step 2, the probe 205′ is cleaved into two pieces, fragment 205 frag which is unbound and washed away in a washing step and 205df which is defanged due to removal of tag 201 (which is washed away as part of unbound fragment 205 frag). Neither of the probe fragments 205df or 205 frag are able to label any biomolecules. The probe 205b is cleaved, but remains attached to the biomolecule of interest by double-crosslinking. In some variations, the probe 205c may be crosslinked to a bait molecule or another proximal biomolecule; however, the principle remains the same. The probe 205b contains tag 201, and as explained in more detail below, labels neighbor molecules.

Labeling system 240 includes labeling complex 208 with label 206 and enzyme or catalyst 207, and enzyme/catalyst substrate 218.

Excess probe is washed away with wash solution and single-crosslinked probes (e.g., in non-lighted areas) are removed through site-specific cleavage as described above. Steps 3 and 4 show labeling of the molecules near the molecule of interest 210 using labeling system 240 shown in FIG. 2B. Other labeling systems can also be used. By way of example, complex 208 conjugates with tag 201, the enzyme or catalyst 207 activates enzyme/catalyst substrate 218 to activated enzyme/catalyst substrate 218′. Since probe 205b is attached to molecule of interest 210, neighbor molecules 211 are labeled, while more distant molecule 231 is not. The cleavable linkers described herein can enable label transfer from the probe to neighbor molecules within a radius of <10 nm (referring to the size of the radius of the trifunctional (multifunctional) probe).

By photoselectively localizing enzyme or catalyst 207, such as peroxidase, near the molecule of interest and labeling the neighbor molecules 211 in the region of interest using the tagging and labeling just described, the coupling reaction can be localized to a region as small as <100 nm. In some variations, a larger region (e.g., up to about 200 nm, up to about 300 nm, up to about 400 nm) could be labeled. Furthermore, some molecules of interest in a sample have more one region of localization and hence interact with different molecular complexes in different locations simultaneously. The light-assisted tag transfer (e.g., tagging neighbor molecules) can be used successively in more than one location. For example, after applying light as shown in FIG. 2B Process C and tagging the neighbor molecules as indicated, the light can be selectively applied to a second (third, fourth, etc.) location in the sample and this process can be repeated as many times as desired. In addition to labeling (depositing labels) to a relatively small number of neighbor molecules in a very small area of a sample, such as due to the use of the microscope analysis to direct the light and the probes described herein, and as explained below, the process can also be performed with sufficiently mild or gentle treatments so that the cell architecture remains intact (e.g., the reactions are also bioorthogonal).

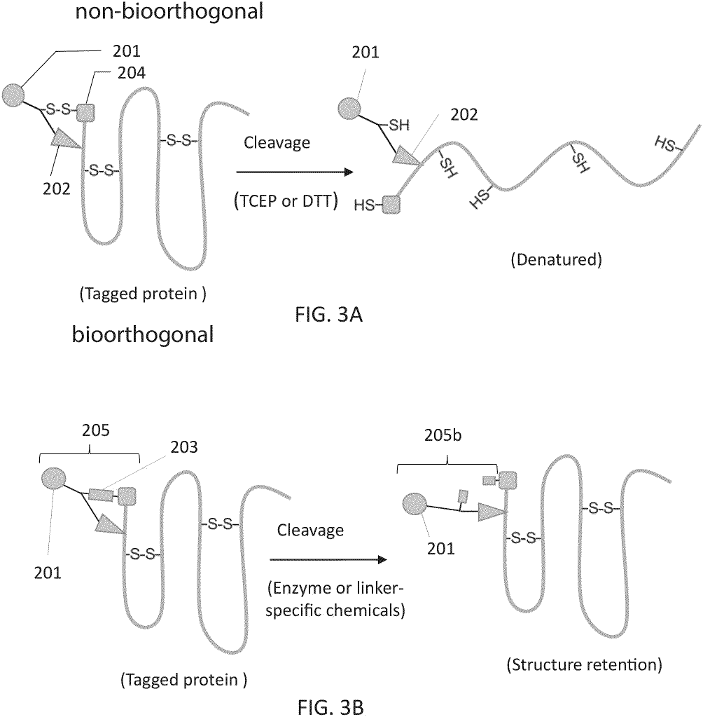

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A). With mild cleavage conditions the protein structure is retained and the reaction is bioorthogonal, while with harsh cleavage conditions, the protein denatures and the reaction is non-bioorthogonal. FIG. 3A schematically illustrates a relatively harsh cleavage, such as one mediated by use of a reducing agent such as tris (2-carboxyethyl) phosphine (TCEP) or dithiothreitol (DTT) in a cleavage reaction. In addition to cleaving the linker, TCEP or DTT break other disulfide bonds, including naturally occurring covalent disulfide bonds commonly found between cysteine amino acids in proteins, denaturing the proteins. It has been estimated that more than 90% of proteins in cells contain at least one cysteine amino acid and that some one-third of the proteins in the eukaryotic proteome form disulfide bonds. Thus, performing a relatively harsh cleavage on a sample to break disulfide bonds is likely to significantly disrupt protein structure, disrupt overall cell architecture, and alter naturally occurring biomolecule interactions. The cleavage reaction can be considered to be a non-bioorthogonal reaction. In some embodiments, a bioorthogonal reaction preserves structures derived from living organisms (e.g., derived from eukaryotes) and excludes consideration of non-living entity structures, such as viruses.

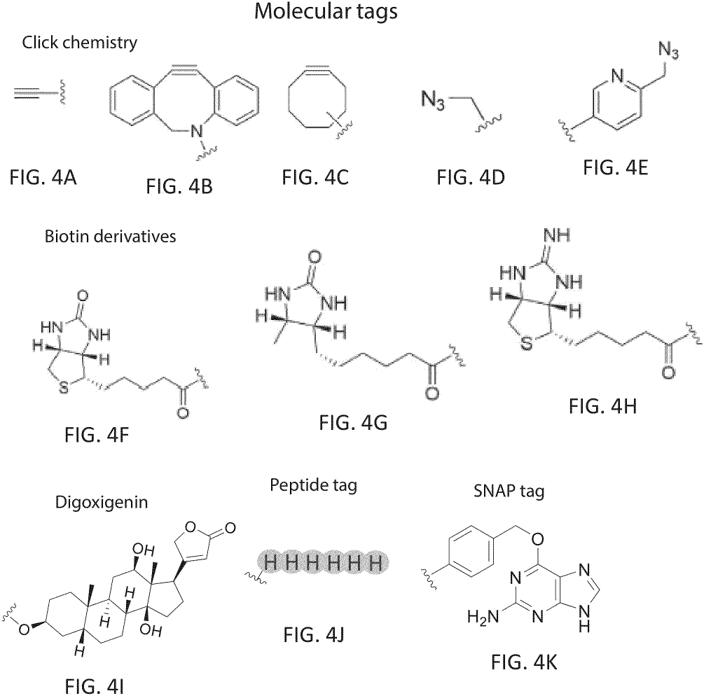

FIG. 4B schematically illustrates a relatively mild cleavage reaction for use with the multifunctional probes described herein. The cleavage reaction uses gentler reagents, such as enzymes or linker-specific chemicals, to cleave the cleavable linker. In some embodiments, mild cleavage reagents are substantially specific. In other words, they substantially and specifically bind to and cleave targets of interest (e.g., the cleavable linker), while substantially not binding to or cleaving other molecules (e.g., less than 1% of the time, less than 0.1%, etc.). In some embodiments, mild cleavage reagents act to cleave other bonds, such as C—C bonds and leave bonds such as disulfide (—S—S—) bonds intact. As illustrated in FIG. 3B, the three-dimensional structure of the protein, mediated by disulfide bonds, remains intact after mild cleavage as described herein. Since tagging and proximity labelling of, for example, naturally occurring neighboring molecules neighboring a protein of interest depends upon the relative proximity of the neighboring molecules to the protein of interest, maintaining the three-dimensional structure of biomolecules and the overall cell architecture can lead to more accurate tagging and labeling of neighboring molecules, reducing both false positives and false negatives in a mild cleavage reaction. The mild cleavage reaction can be bioorthogonal in that it does not substantially disrupt naturally occurring protein structure or cell architecture.

FIGS. 4A-4K show examples of tags that can be used in the photoreactive and cleavable probes described herein. The tags are configured to interact with a detectable label to label biomolecules neighboring a target molecule of interest. FIG. 4A-FIG. 4E show examples of click chemistry tags that can be used with the probes. The click chemistry tag may be, for example, an azide moiety or an alkyne moiety. FIGS. 4F-4H show examples of biotin derivatives that can be used with as probe tags. FIG. 4I shows a digoxigenin moiety tag. FIG. 4J shows a peptide tag. In particular, FIG. 4J shows a poly His tag with 6 histidines (SEQ ID NO:1). However, a histidine tag could instead fewer or more histidines, such as 5 (SEQ ID NO: 17) or 7-10 or more (SEQ ID NO: 18). FIG. 4K shows a SNAP-tag. FIG. 6K shows a SNAP-tag and a CLIP-tag or HaloTag could also be used.

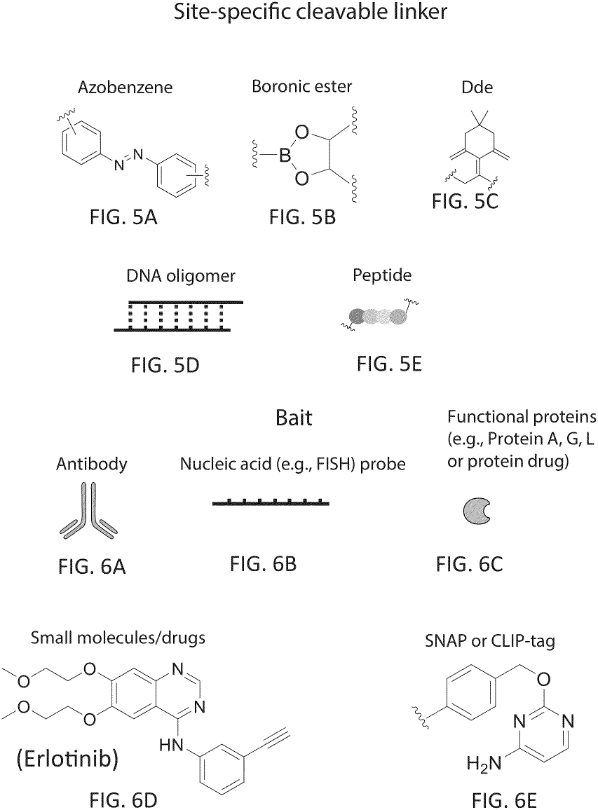

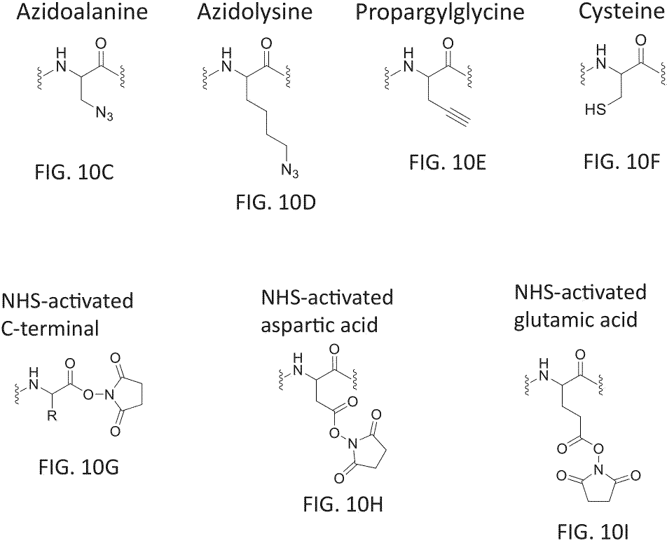

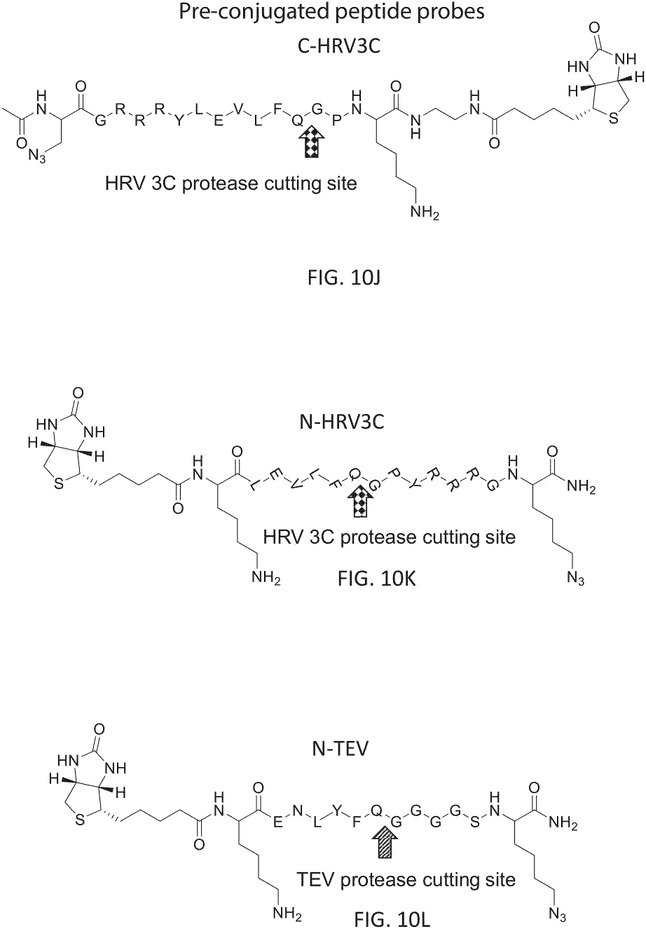

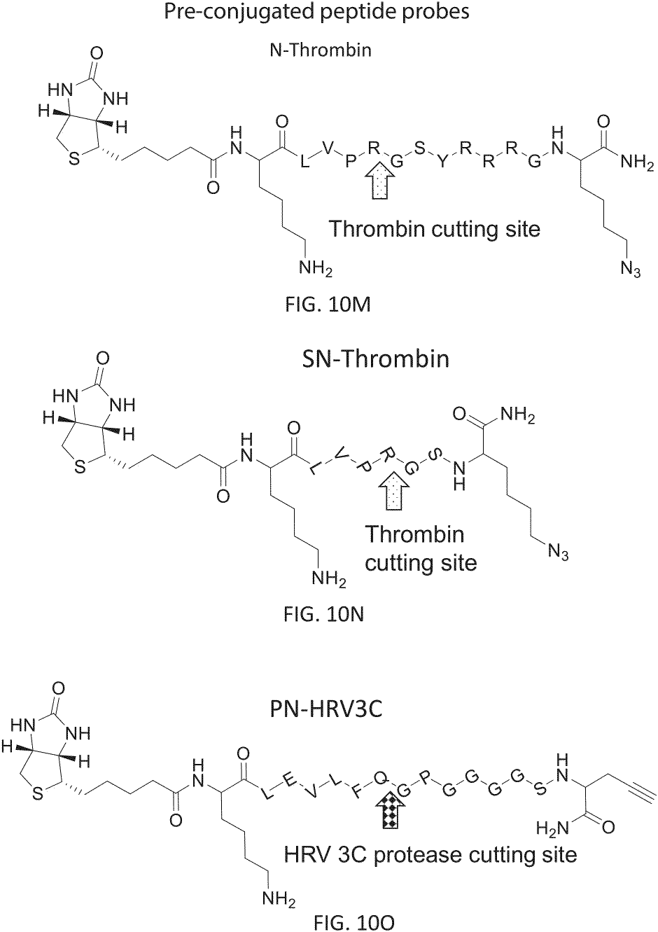

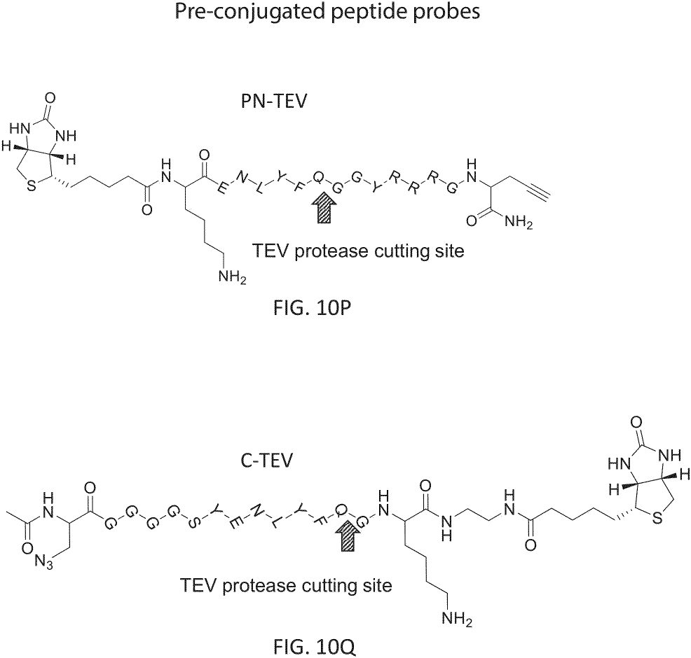

FIGS. 5A-5E show examples of site-specific cleavable linkers that can be used in the photoreactive and cleavable probes described herein. FIG. 5A shows an azobenzene moiety. An azobenzene linker can be cleaved during the cleavage step such as with sodium dithionite or azoreductase. FIG. 5B shows a boronic ester moiety. A boronic ester cleavable linker can be cleaved with thionyl chloride and pyridine. FIG. 5C shows a Dde moiety. The Dde cleavable linker can be cleaved using enzymes or simple small molecules. FIG. 5D shows a DNA oligomer cleavable linker and other nucleic acid molecules can instead be used. DNA oligomers can be cleaved using restriction enzymes, nucleases, or competitive methods using complementary oligomers, depending upon what molecule is labeled. FIG. 5E shows a peptide moiety linker and peptide moiety linkers are discussed below in more detail in reference to FIG. 10A-FIG. 10Q. A peptide linker can be cleaved during the cleavage step using a protease. In some embodiments, a site-specific cleavable linker can be conjugated to a bait molecule. For example, a linker conjugating to bait molecules such as NHS-esters can bind to protein baits, such as antibodies. A particular cleavage linker and associated cleavage reagent can be chosen for various reasons, such as cost or cleavage efficiency.

FIGS. 6A-6E shows examples of bait molecules that can be used in the photoreactive and cleavable probes described herein to conjugate with a molecule of interest in a sample. FIG. 6A shows an antibody that can be used a bait molecule. Any time of antibody can be used. FIG. 6B shows a nucleic acid portion that can be used as a bait molecule, such as fluorescent in situ hybridization probe (FISH probe). FIG. 6C shows a representation of a functional protein that can be used as a bait molecule. Examples of functional proteins include Protein A, Protein G, Protein L, protein A/G, or a protein drug. Other bait molecules that can be used in the photoreactive and cleavable probes described herein include biologic drugs. Examples of biologic drugs that can be used as bait include abatacept (Orencia); abciximab (ReoPro); abobotulinumtoxinA (Dysport); adalimumab (Humira); adalimumab-atto (Amjevita); ado-trastuzumab emtansine (Kadcyla); aflibercept (Eylea); agalsidase beta (Fabrazyme); albiglutide (Tanzeum); aldesleukin (Proleukin); alemtuzumab (Campath, Lemtrada); alglucosidase alfa (Myozyme, Lumizyme); alirocumab (Praluent); alteplase, cathflo activase (Activase); anakinra (Kineret); asfotase alfa (Strensiq); asparaginase (Elspar); asparaginase erwinia chrysanthemi (Erwinaze); atezolizumab (Tecentriq); basiliximab (Simulect); becaplermin (Regranex); belatacept (Nulojix); belimumab (Benlysta); bevacizumab (Avastin); bezlotoxumab (Zinplava); blinatumomab (Blincyto); brentuximab vedotin (Adcetris); canakinumab (Ilaris); capromab pendetide (ProstaScint); certolizumab pegol (Cimzia); cetuximab (Erbitux); collagenase (Santyl); collagenase clostridium histolyticum (Xiaflex); daclizumab (Zenapax); daclizumab (Zinbryta); daratumumab (Darzalex); darbepoetin alfa (Aranesp); denileukin diftitox (Ontak); denosumab (Prolia, Xgeva); dinutuximab (Unituxin); dornase alfa (Pulmozyme); dulaglutide (Trulicity); ecallantide (Kalbitor); eculizumab (Soliris); elosulfase alfa (Vimizim); elotuzumab (Empliciti); epoetin alfa (Epogen/Procrit); etanercept (Enbrel); etanercept-szzs (Erelzi); evolocumab (Repatha); filgrastim (Neupogen); filgrastim-sndz (Zarxio); follitropin alpha (Gonal f); galsulfase (Naglazyme); glucarpidase (Voraxaze); golimumab (Simponi); golimumab injection (Simponi Aria); ibritumomab tiuxetan (Zevalin); idarucizumab (Praxbind); idursulfase (Elaprase); incobotulinumtoxinA (Xeomin); infliximab (Remicade); infliximab-dyyb (Inflectra); interferon alfa-2b (Intron A); interferon alfa-n3 (Alferon N Injection); interferon beta-1a (Avonex, Rebif); interferon beta-1b (Betaseron, Extavia); interferon gamma-1b (Actimmune); ipilimumab (Yervoy); ixekizumab (Taltz); laronidase (Aldurazyme); mepolizumab (Nucala); methoxy polyethylene glycol-epoetin beta (Mircera); metreleptin (Myalept); natalizumab (Tysabri); necitumumab (Portrazza); nivolumab (Opdivo); obiltoxaximab (Anthim); obinutuzumab (Gazyva); ocriplasmin (Jetrea); ofatumumab (Arzerra); olaratumab (Lartruvo); omalizumab (Xolair); onabotulinumtoxinA (Botox); oprelvekin (Neumega); palifermin (Kepivance); palivizumab (Synagis); panitumumab (Vectibix); parathyroid hormone (Natpara); pegaspargase (Oncaspar); pegfilgrastim (Neulasta); peginterferon alfa-2a (Pegasys); peginterferon alfa-2b (PegIntron, Sylatron); peginterferon beta-1a (Plegridy); pegloticase (Krystexxa); pembrolizumab (Keytruda); pertuzumab (Perjeta); ramucirumab (Cyramza); ranibizumab (Lucentis); rasburicase (Elitek); raxibacumabreslizumab (Cinqair); reteplase (Retavase); rilonacept (Arcalyst); rimabotulinumtoxinB (Myobloc); rituximab (Rituxan); romiplostim (Nplate); sargramostim (Leukine); sebelipase alfa (Kanuma); secukinumab (Cosentyx); siltuximab (Sylvant); tbo-filgrastim (Granix); tenecteplase (TNKase); tocilizumab (Actemra); trastuzumab (Herceptin); ustekinumab (Stelara); vedolizumab (Entyvio); ziv-aflibercept (Zaltrap).

FIG. 6D also shows small molecules/drugs can be used as bait molecules. By way of example, erlotinib is shown. FIG. 6E shows a CLIP-tag and other members of self-labeling moieties could be used (e.g., HaloTag or SNAP-Tag).

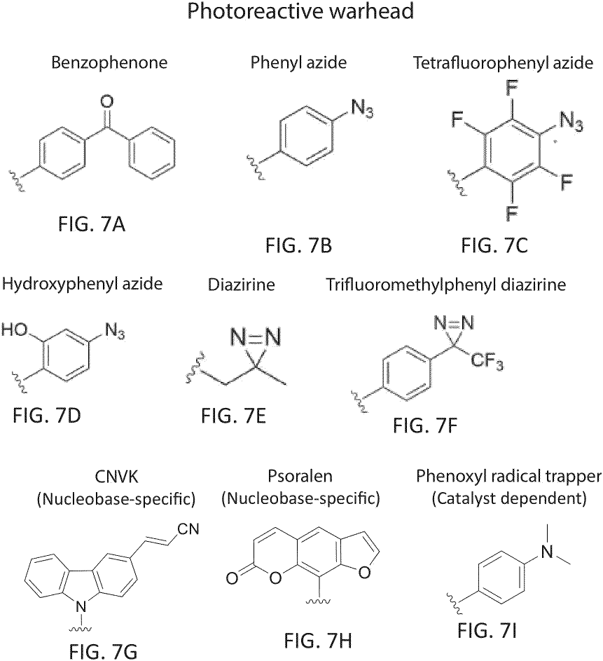

FIGS. 7A-7I show examples of photoactive warheads that can be used in the photoreactive and cleavable probes described herein. FIG. 7A shows a benzophenone photoactive warhead, which can be activated by either 320-365 nm UV-A irradiation of single photon excitation or 720-800 nm of two photon excitation. FIGS. 7B, 7C and 7D shows aryl azide-based warheads which can be activated by either 250-365 nm irradiation of single photon excitation or 800 nm of two photon excitation. FIG. 7B shows phenyl azide photoactive warheads. FIG. 7C shows tetrafluorophenyl azide photoactive warheads. FIG. 7D shows hydroxyphenyl azide photoactive warheads. FIG. 7E shows diazirine photoactive warheads. FIG. 7F shows trifluoromethylphenyl diazirine photoactive warheads. FIG. 7G shows 3-cyanovinylcarbazole nucleoside (CNVK) photoactive warheads which is nucleobase specific. FIG. 7H shows psoralen photoactive warheads which is also nucleobase specific. Psoralens react with DNA or RNA to form covalent adducts. In some embodiments, psoralen photoactive warheads can be activated by long wavelength US light (e.g., UVA, 310-400 nm). FIG. 7I shows phenoxyl radical trapper photoactive warheads which is catalyst dependent. The selection of a particular light-activated warhead can depend on the desired wavelength and the types of the bait molecule. For example, the constituents of the multifunctional probe and constituents for the pre-probe analysis can be chosen so as to not interfere (or minimally interfere) with each other.

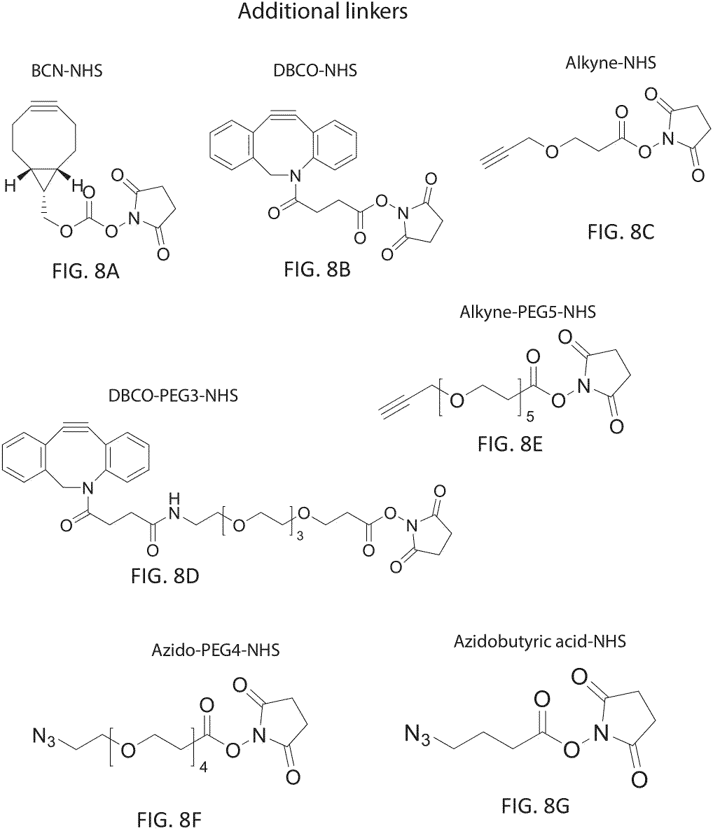

FIGS. 8A-8G show additional examples of linkers that can be used as linkers in the photoreactive and cleavable probe described herein. FIG. 8A shows a BCN-NHS linker. FIG. 8B shows DBCO-NHS linker. FIG. 8C shows Alkyne-NHS linker. FIG. 8D shows DBCO-PEG3-NHS linker. FIG. 8E shows Alkyne-PEG5-NHS linker. FIG. 8F shows Azido-PEG4-NHS linker. FIG. 8G shows azidobutyric acid-NHS linker.

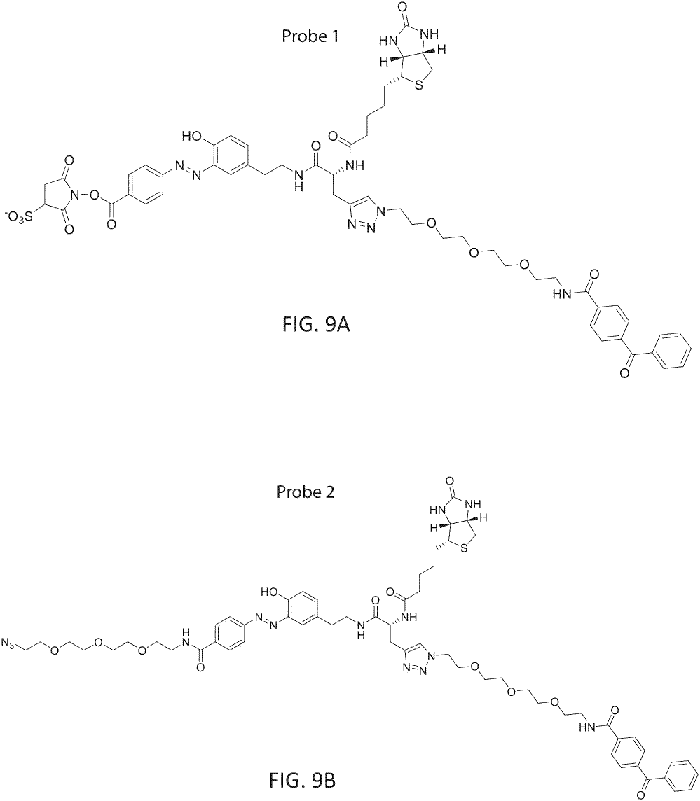

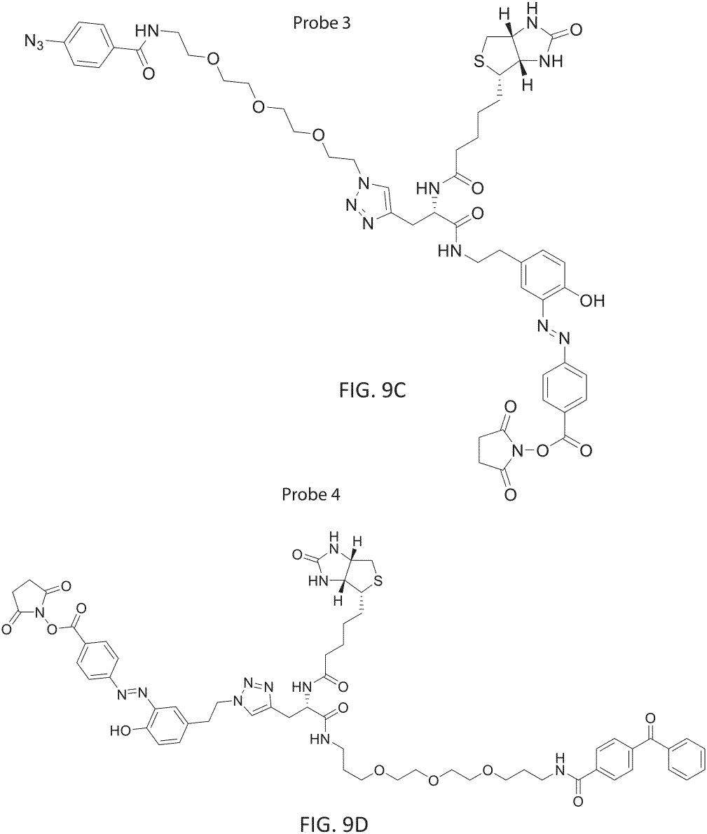

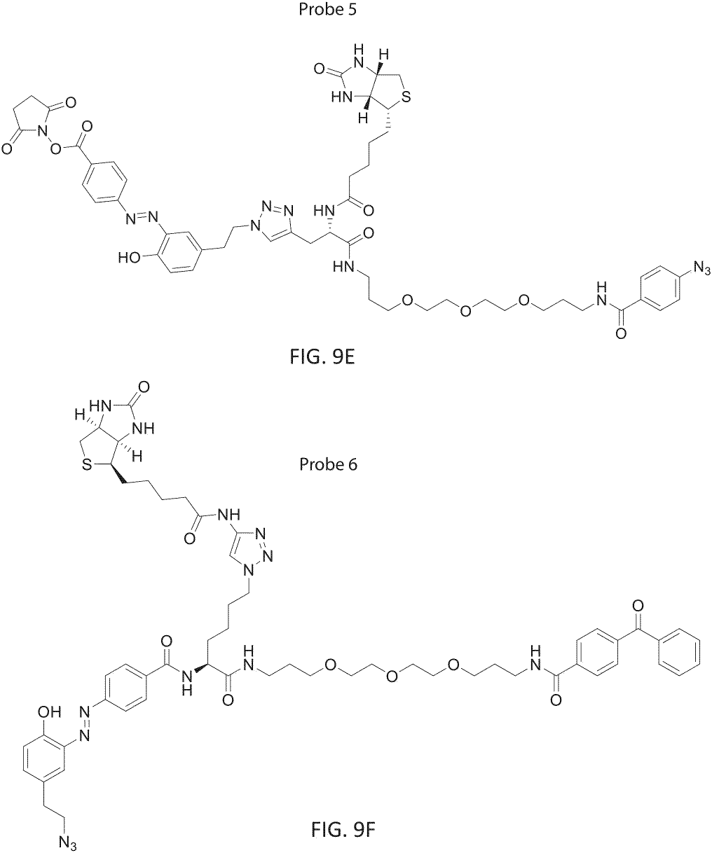

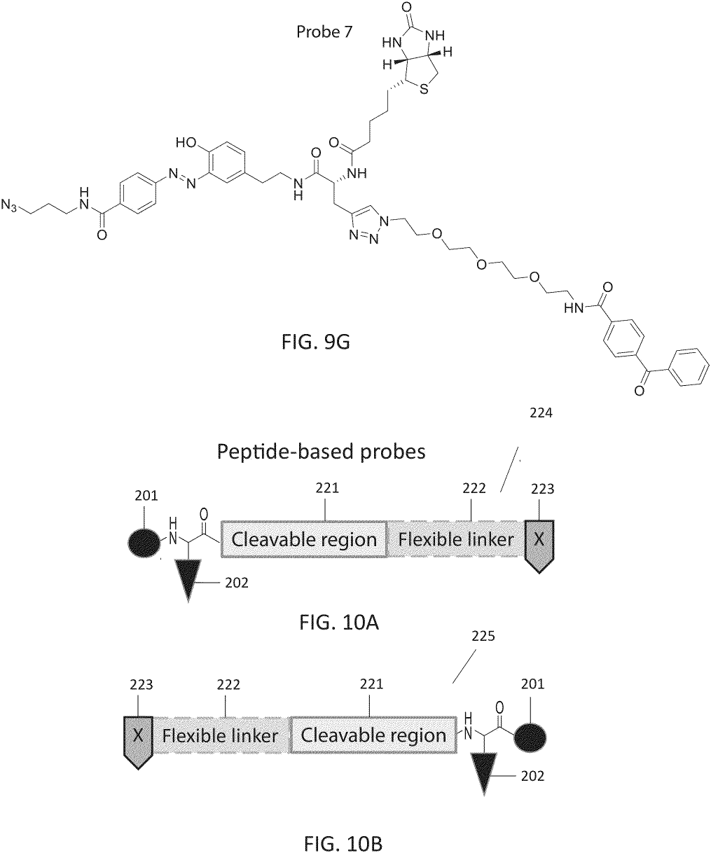

FIGS. 9A-9G show examples of photoreactive and cleavable probes that can be used in the compositions and for practicing the methods described herein. The probes have multivalent cores with a plurality of attachment sites. A tag, a cleavable linker, and a light activated warhead are bound to the attachment sites on the probe. In some embodiments, the multivalent core includes the moiety of formula (I). In some embodiments, n is 1, 2, 3, 4, 5, or 6. In some embodiments, R1 and R2 each independently are hydrogen, substituted, alkyl, substituted alkenyl, substituted alkynyl, substituted carbocyclyl, substituted heterocyclyl, substituted aryl, substituted heteroaryl, or a nitrogen protecting group. In some embodiments one of R3 and R4 is - (CH2)x(OCH2CH2)y(CH2)zNR5R6, and the other is an attachment site, wherein x is 1, 2, 3, 4, 5, or 6; y is 1, 2, 3, 4, 5, or 6; z is 0, 1, 2, 3, 4, 5, or 6; and one of R5 and R6 is an attachment site, and the other is hydrogen, substituted alkyl, substituted alkenyl, substituted alkynyl, substituted carbocyclyl, substituted heterocyclyl, substituted aryl, substituted heteroaryl, or a nitrogen protecting group.

FIGS. 10A-10B schematically illustrate peptide-based photoreactive and cleavable probes. These probes have a peptide region cleavable by a peptide cleavage reagent, such as by a protease that recognizes a specific peptide sequence and the peptide regions are specifically cleavable (e.g., by a protease). FIG. 10A shows an example of a peptide-based probe 224 with tag 201 and photoreactive warhead 202 on the N-terminal end of the peptide region. FIG. 10B shows an example of a peptide-based probe 225 with tag 201 and warhead 202 on the C-terminal end of the peptide region. FIGS. 10A-10B also show probes with an additional, flexible linker 222 (also referred to herein as a spacer) and an optional clickable amino acid 223. FIGS. 10C-10I show examples of reactive or clickable amino acids that can be used with the probes shown in FIGS. 10A and 10B. FIG. 10C shows azidoalanin clickable amino acid. FIG. 10D shows azidolysine clickable amino acid. FIG. 10E shows propargylglycine clickable amino acid. FIG. 10F shows cysteine clickable amino acid. FIG. 10 G shows NHS-activated C-terminal clickable amino acid. FIG. 10 H shows NHS-activated aspartic acid clickable amino acid. FIG. 10I shows NHS-activated NHS-activated glutamic acid.

FIGS. 10J-10Q show examples of peptide-based photoreactive and cleavable probes schematically illustrated in FIGS. 10A-10B. The cleavage sites for the human rhinovirus 3C (HRV 3C) protease (checkered arrow), tobacco etch virus (TEV) protease (striped arrow), and thrombin (dotted arrow) are indicated. The proteolytically cleavable peptide sequences can be specifically cleaved by a protease during the cleavage step. Examples of proteolytically cleavable peptide sequences that can be used with the probes described herein include those recognized by activated blood coagulation factor X enteropeptidase (also referred to herein as factor X enteropeptidase or factor Xa), human rhinovirus (HRV) 3C protease, thrombin, and tobacco etch virus (TEV) protease. Of these proteases, the factor Xa and thrombin are naturally found in blood. These proteases may recognize and cleave proteins in a cell or cell extract other than the proteolytically cleavable peptide sequences of the peptide-based probes. Although in some cases, this may upset the naturally occurring protein environment in the samples and lead to misleading or artefactual results in some analyses, it is also noted that the number of these cleavage reactions may be sufficiently limited so as to be useful for certain purpose or in certain situations. Cleavage reactions that do not interfere with naturally occurring biomolecules (e.g., naturally occurring proteins in a cell or tissue sample) are considered bioorthogonal and probes cleavable under circumstances that maintain naturally occurring protein structure can be considered to be a bioorthogonally cleavable probe with a bioorthogonally cleavable peptide sequence. As discussed above, while the Sulfo-SBED probe may find use with certain of the methods described herein for particular applications, in other embodiments, the cleavage of the Sulfo-SBED probe with dithiothreitol (DTT) or 2-mercaptoethanol to cleave its S-S bond also undesirably disrupts naturally occurring proteins (e.g., it is non-bioorthogonal).

Enterokinase recognizes the peptide sequence DDDDK| (SEQ ID NO: 2) for cleavage, where cleavage occurs in the linker bond after the lysine.

Factor Xa recognizes the peptide sequence LVPR|GS (SEQ ID NO:3) for cleavage, where cleavage occurs in the linker bond between the arginine and the glycine.

Human rhinovirus (HRV) 3C protease recognizes the peptide sequence LEVLFQ|GP (SEQ ID NO:4) for cleavage, where cleavage occurs in the linker bond between the glutamine and the glycine.

TEV protease prefers the peptide sequence ENLYFQ|S (SEQ ID NO:5) for cleavage, where cleavage occurs in the linker bond between the glutamine and the serine. TEV protease can also recognize the sequence ENLYFQ|G (SEQ ID NO:6) for cleavage, where cleavage occurs between the linker bond between the glutamine and the glycine.

Thrombin recognizes the peptide sequence LVPR|GS (SEQ ID NO:7) for cleavage, where cleavage occurs in the linker bond between the arginine and the glycine

In addition to specific recognition sequences for proteolysis, the peptide portion may contain additional amino acids. Photoreactive and cleavable probes can have either C-terminal or N-terminal tags (e.g., biotinylation).

FIG. 10J shows a C-HRV3C pre-conjugated peptide probe with an HRV 3C proteolytically cleavable peptide sequences GRRRYLEVLFQGP (SEQ ID NO: 8).

FIG. 10K shows an N-HRV3C pre-conjugated peptide probe with an HRV 3C protease cutting site peptide sequence LEVLFQGPYRRRG (SEQ ID NO: 9).

FIG. 10L shows an N-TEV pre-conjugated peptide probe with a TEV protease cutting site peptide sequence ENLYFQGGGGS (SEQ ID NO: 10).

FIG. 10M shows an N-Thrombin pre-conjugated peptide probe with a thrombin protease cutting site peptide sequence LVPRGSYRRRG (SEQ ID NO: 11).

FIG. 10N shows SN-Thrombin conjugated peptide probe with a thrombin protease cutting site peptide sequence LVPRGS (SEQ ID NO: 12).

FIG. 10O shows PN-HRV3C conjugated peptide probe with HRV 3C protease cutting site peptide sequence LEVLFQGPGGGGS (SEQ ID NO: 13).

FIG. 10P shows a PN-TEV conjugated peptide probe with a TEV protease cutting site peptide sequence ENLYFQGGYRRRG (SEQ ID NO: 14).

FIG. 10Q shows a C-TEV conjugated peptide probe with a TEV protease cutting site peptide sequence GGGGSYENLYFQG (SEQ ID NO: 15).

As indicated above, some probes include a flexible linker (also referred to herein as a spacer). Flexible linkers are flexible molecules or stretches of molecules that are used to link two molecules or moieties together. Linkers may be composed of flexible groups so that adjacent domains are free to move relative to another. Flexible linkers may include flexible amino acid residues, such as glycine (G) or serine (S). Flexible linkers may also include threonine (T) and alanine (A) residues. A string of amino acids can be repeated in the linker. For example, a linker may include a length of glycine residues followed by a serine residue, such as forming an (GGGGS)n oligomer, where n is 1, 2, 3, 4, 5, 6, 7, 8 or larger (SEQ ID NO: 19) and the GGGGS motif (SEQ ID NO: 16) is repeated. Flexible linkers can also include alkyl groups, such as a polyethylene glycol (CH₂CH₂O)ₘ linker, where m is from 1 to 50, or 2-30, or 3-6. Other examples of polymeric flexible linkers include polypropylene glycol, polyethylene, polypropylene, polyamides, and polyesters. Flexible linkers can be linear molecules in a chain of at least one or two atoms and can include more.

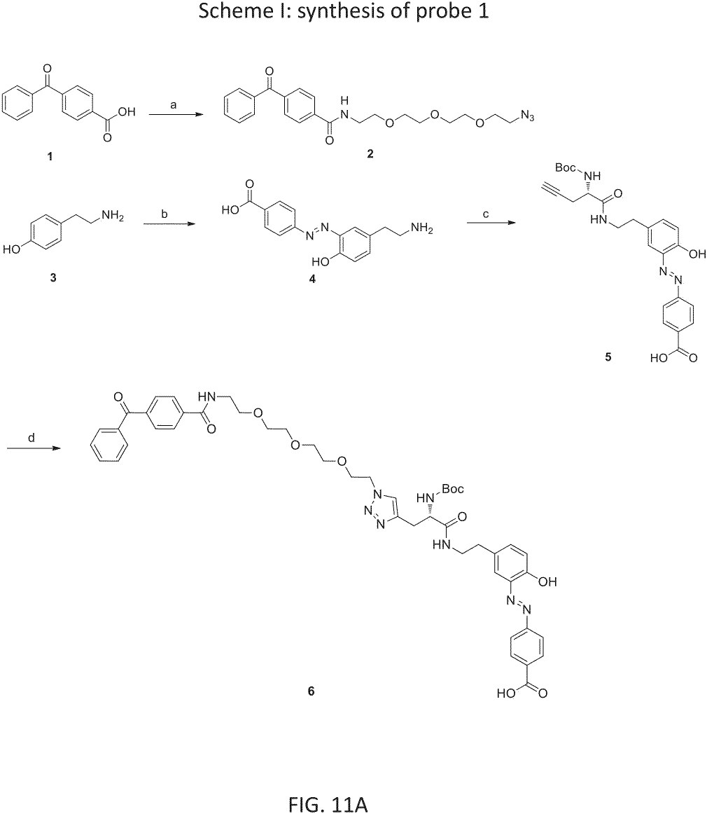

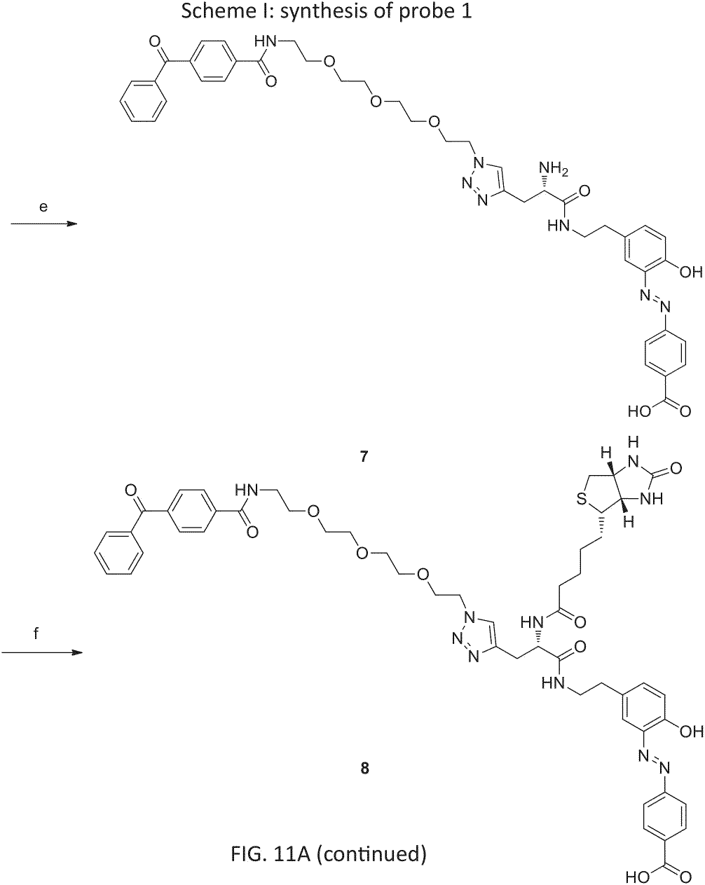

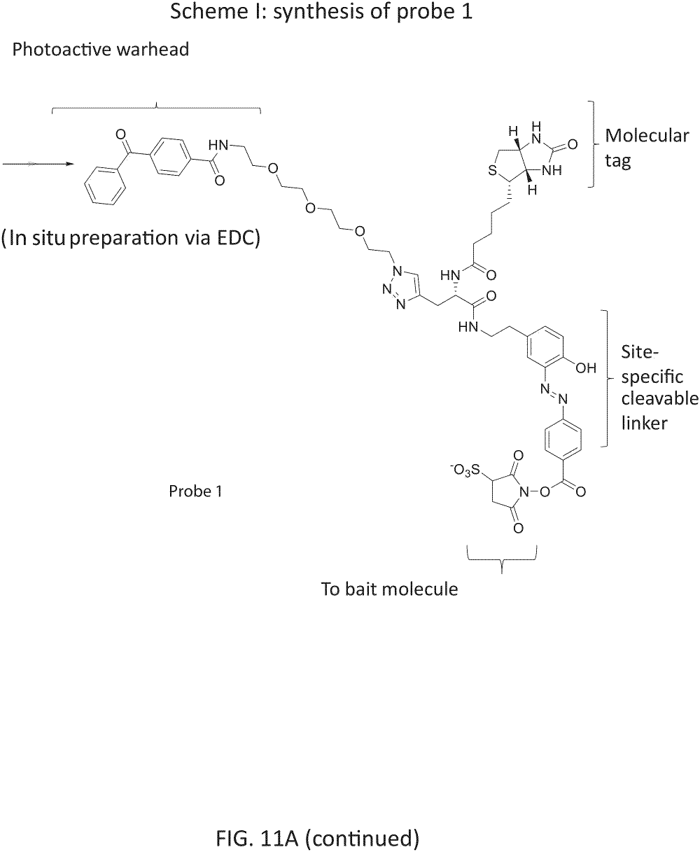

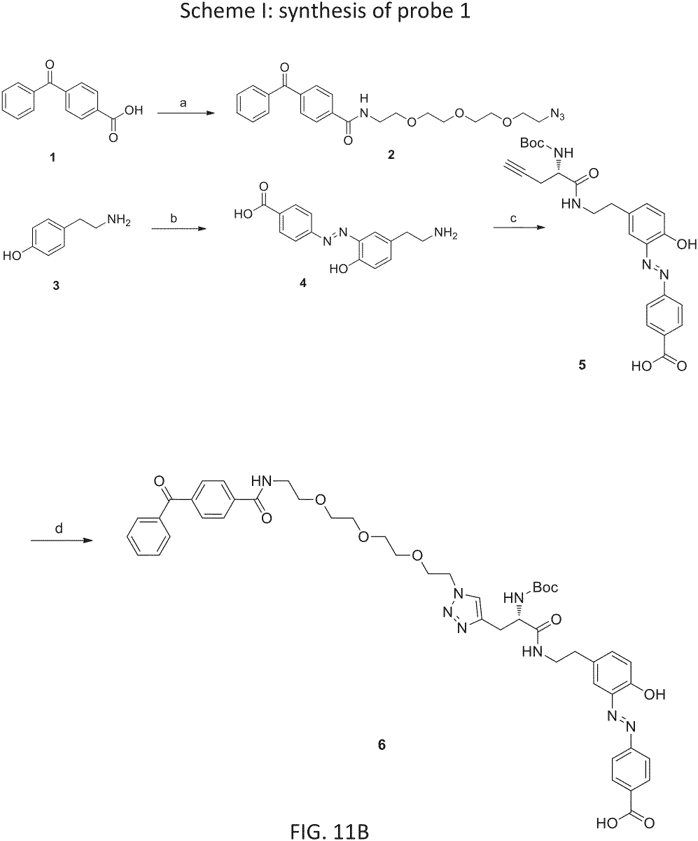

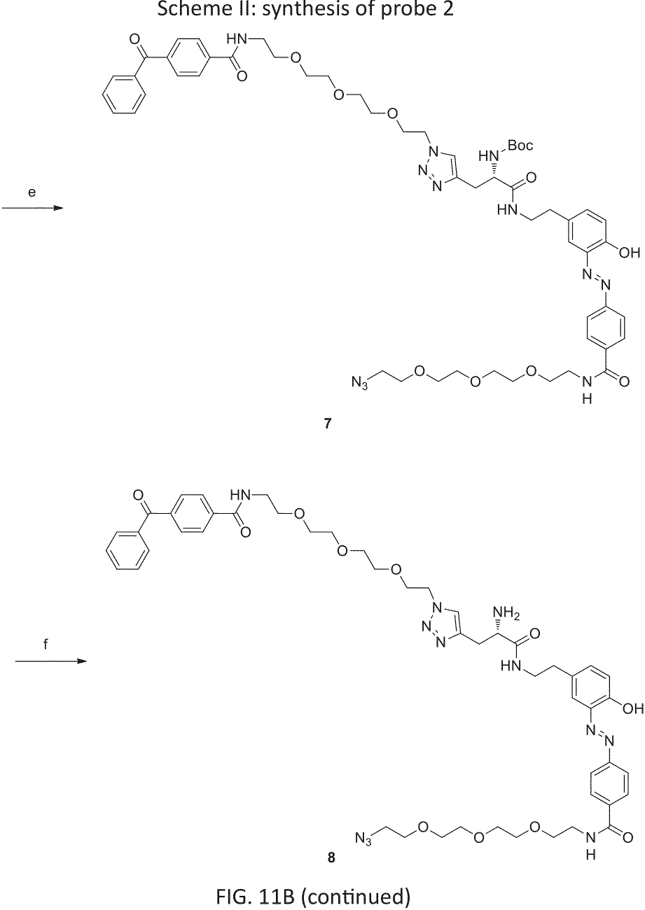

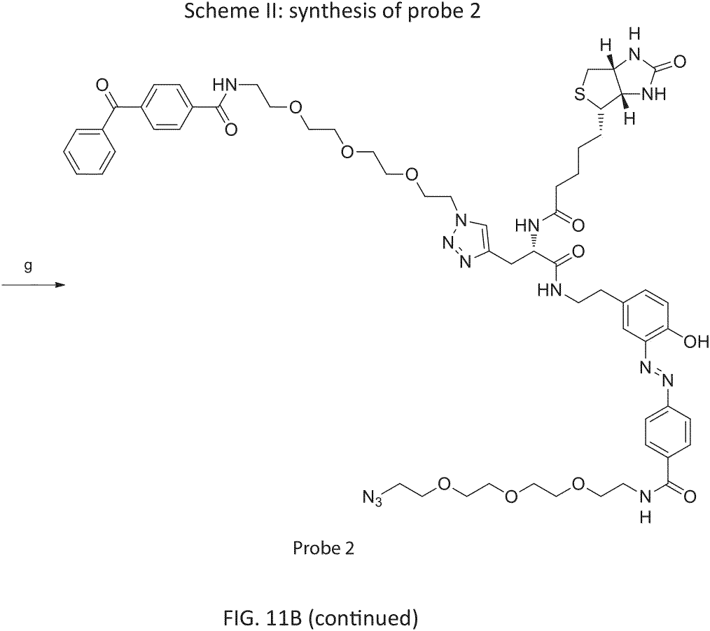

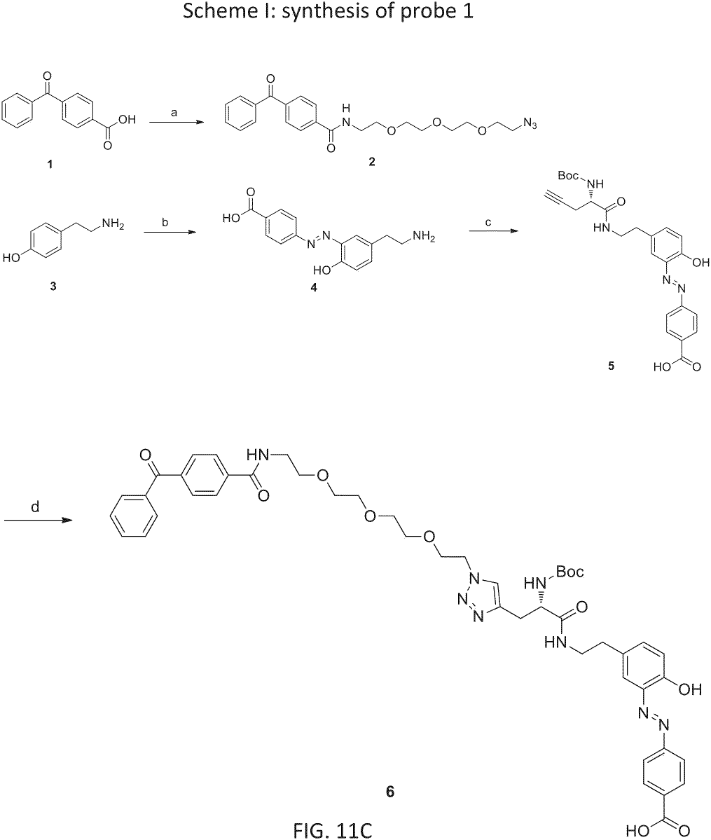

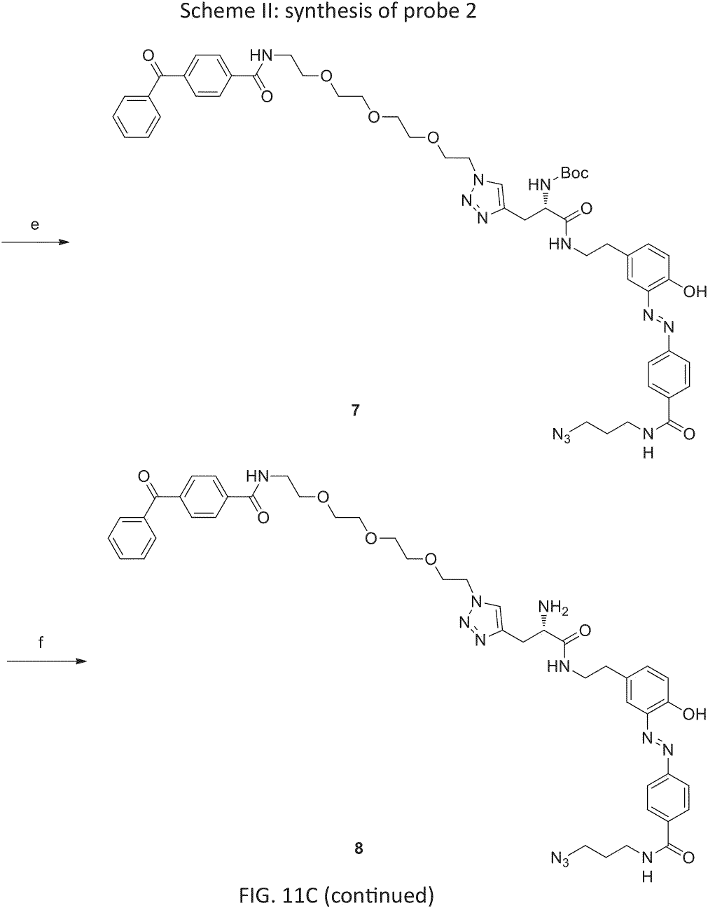

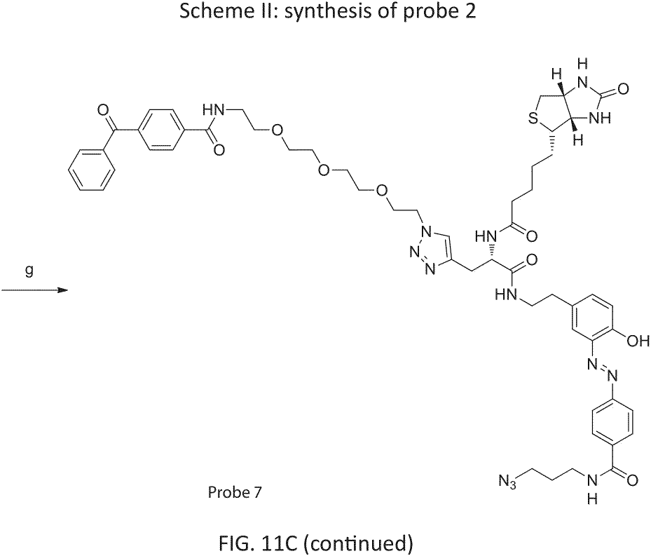

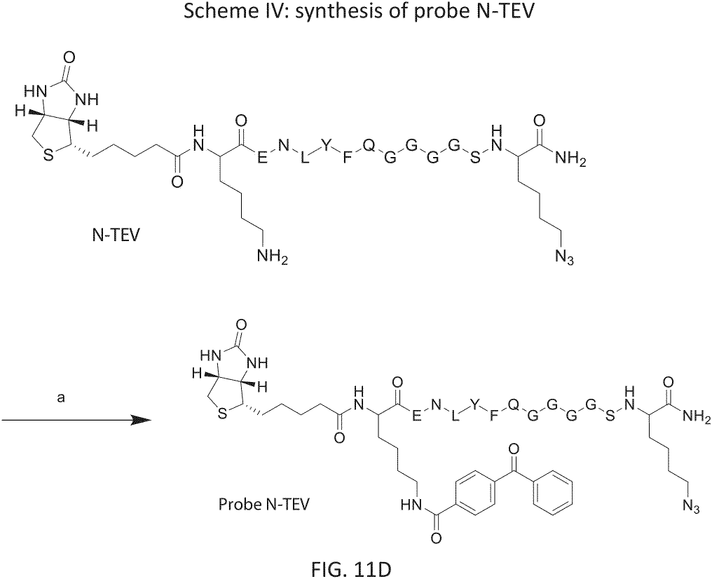

FIGS. 11A-11D illustrate methods to synthesize the photoreactive and cleavable probes described herein. The methods create probes with a tag, a cleavable linker, and a light activated warhead. The figures also illustrate regions where bait molecules can be conjugated. The trifunctional molecular probes can be synthesized by using commercially available molecules as building blocks and regular-used synthesis steps. The schemes shown in FIGS. 11A-11D for synthesis of the probes are given as examples and not for limiting purposes. FIG. 11A shows a synthesis scheme for probe 1. FIG. 11B shows a synthesis scheme for probe 2. FIG. 11C shows a synthesis scheme for probe 7. FIG. 11D shows a synthesis scheme for Probe IV N-TEV.

Some embodiments provide a photoreactive and cleavable probe including a multivalent core comprising a plurality of attachment sites. Some embodiments provide a tag bound to one of the attachment sites, wherein the tag is configured to conjugate to a label. Some embodiments provide a cleavable linker bound to a second of the attachment sites and configured to link to a bait molecule, wherein the cleavable linker includes a peptide sequence.

Some embodiments provide a light-activated warhead bound to a third of the attachment sites, wherein the multivalent core includes the moiety of formula (II) or (III):

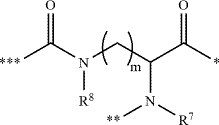

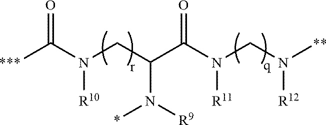

wherein m, r and q each independently are 1, 2, 3, 4, 5, or 6; wherein * comprises an attachment site of one of the plurality of attachment sites for the cleavable linker, wherein ** includes a different attachment site of the plurality of attachment sites for one of either the tag or the photoreactive warhead; wherein *** includes a different attachment site of the plurality of attachment sites for either the photoreactive warhead or the tag, respectively, and R7, R8, R9, R10, R11, and R12 each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In some embodiments of the the photoreactive and cleavable probe wherein ** includes the attachment site for the tag, and *** includes the attachment site for the photoreactive warhead.

In some embodiments of the photoreactive and cleavable probe, the peptide sequence includes a protease recognition sequence.

In some embodiments of the photoreactive and cleavable probe, wherein the peptide sequence comprises a human rhinovirus 3C (HRV 3C) protease recognition sequence, a tobacco etch virus (TEV) protease recognition sequence, or a thrombin recognition sequence.

In some embodiments of the photoreactive and cleavable probe, the cleavable linker further includes a conjugatable amino acid.

In some embodiments of the photoreactive and cleavable probe, the cleavable linker further includes a cysteine or clickable amino acid amino acid.

In some embodiments of the photoreactive and cleavable probe, the cleavable linker comprises a clickable amino acid with an azido or alkyne moiety.

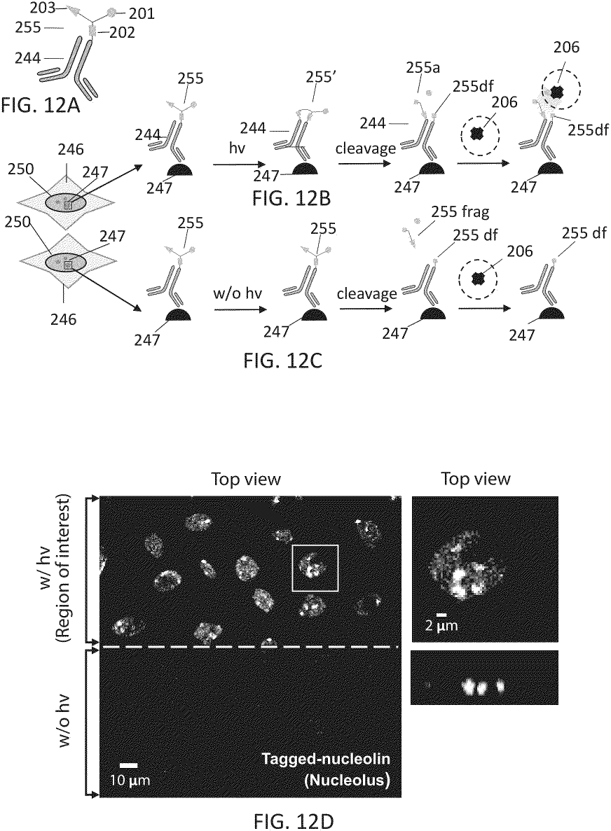

FIG. 12A schematically illustrates a photoreactive and cleavable probe conjugated to an antibody bait. FIG. 12B and FIG. 12C schematically illustrates a reaction scheme for performing photoselective tagging of a molecule using a photoreactive and cleavable probe conjugated to an antibody bait for labeling proteins in the cell nucleolus. FIG. 12B illustrates how the reaction proceeds using controlled light. FIG. 12B also illustrates how the cleavable probes are cleaved to reduce background in non-lighted areas. The reaction shown in FIG. 12B is similar as to that shown in FIG. 2C except that bait molecule 204 is an antibody 244 and the probe 255 includes the antibody 244 as bait. When the photoreactive warhead of the probe 255 is activate, it binds to the bait/antibody 244, rather than a cellular molecule. However, the probe 255 is still retained in the vicinity of the protein of interest, in this case nucleolin in nucleolus 247 in the nucleus 250 of cell 246. During the cleavage step, probe 255 out of the photoselected area is still cleaved into fragment 255 frag and defanged probe fragment 255df which are washed away during a wash step. FIG. 12D shows results from using the reaction schemes shown in FIG. 12A and FIG. 12B. The nucleolin protein is specifically tagged in the presence of light (top and right panels), but is not tagged in the absence of light (bottom panel). Probes linked to bait molecules are selectively retained through light-activation followed by cleavage and conjugated to enzymes (e.g., HRP in this example) for spatial labeling at a radius from about 10 nm to about 100 nm, depending upon the particular enzymes and reaction times used. In some embodiments of the method, selectively illuminating includes illuminating a zone defined by point spread function.

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and layering can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halo-tag, SNAP-tag). In some embodiments a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subject to fixation followed by embedding, such as formalin fixation and paraffin embedding (FFPE).

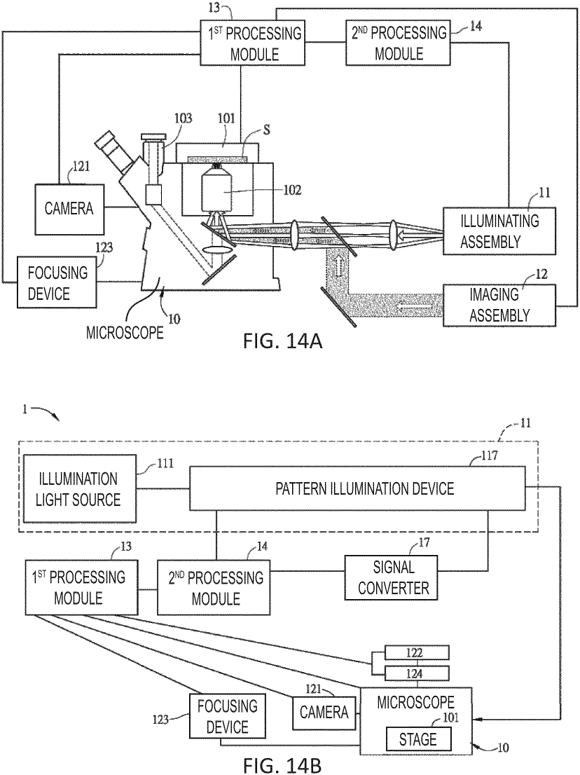

This disclosure provides an embodiment which is also a microscope-based system for image-guided microscopic illumination. Please refer to FIGS. 14A and 14B. The microscope-based system of this embodiment comprises a microscope 10, an imaging assembly 12, an illuminating assembly 11, and a processing module 13a. The microscope 10 comprises an objective 102 and a stage 101. The stage 101 is configured to be loaded with a sample S. The imaging assembly 12 may comprise a (controllable) camera 121, an imaging light source 122, a focusing device 123, and a first shutter 124. Please further refer to FIG. 14B, the illuminating assembly 11 may comprise an illumination light source 111 and a pattern illumination device 117. The pattern illumination device 117 may include a second shutter 112, a lens module 113 (such as the relay lens 113a and 113b, a quarter wave plate 113c), at least a pair of scanning mirrors 115 and a scan lens 116. Alternatively, digital micromirror device (DMD) or spatial light modulator (SLM) can be used as the pattern illumination device 117.

In this embodiment, the processing module 13a is coupled to the microscope 10, the imaging assembly 12, and the illuminating assembly 11. The processing module 13a can be a computer, a workstation, or a CPU of a computer, which is capable of executing a program designed for operating this system.

The processing module 13a controls the imaging assembly 12 such that the camera 121 acquires at least one image of the sample S of a first field of view, and the image or images are transmitted to the processing module 13a and processed by the processing module 13a automatically in real-time based on a predefined criterion, so as to determine an interested region in the image S and so as to obtain a coordinate information regarding to the interested region. Later, the processing module 13a may control the pattern illumination device 117 of the illuminating assembly 11 to illuminate the interested region of the sample S according to the received coordinate information regarding to the interested region. Also, after the interested region is fully illuminated, the processing module 13a controls the stage 101 of the microscope 10 to move to a second field of view which is subsequent to the first field of view.

In this embodiment, the imaging light source 122 provides an imaging light through an imaging light path to illuminate the sample S during imaging the sample. The first shutter 124, along the imaging light path, is disposed between the image light source 122 and the microscope 10. The controllable camera 121 is disposed on the microscope 10 or on the imaging light path.

Also, the illuminating light source 111 provides an illuminating light through an illuminating light path to illuminate the sample S. The pattern illumination device 117, along the illuminating light path, is disposed between the illumination light source 111 and the microscope 10.

This disclosure provides another embodiment which is also a microscope-based system for image-guided microscopic illumination. This system includes an additional processing module to improve illumination performance and will be describe in detail. Please refer to FIGS. 14A and 14B. FIG. 14A represents a schematic diagram of an imaging-guided system according to one embodiment of the present disclosure, and FIG. 14B depicts the optical path of the image-guided system of FIG. 14A.

As shown in FIGS. 14A and 14B, the microscope-based system 1 for image-guided microscopic illumination comprises a microscope 10, an illuminating assembly 11, an imaging assembly 12, a first processing module 13 and a second processing module 14. The microscope-based system 1 is designed to take a microscope image or images of a sample and use this image or these images to determine and shine an illumination pattern on the sample, finishing all steps for one image rapidly (e.g. within 300 ms), and within a short time (e.g. 10 hours) for the entire illumination process for a proteomic study.

The microscope 10 comprises a stage 101, an objective 102 and a subjective 103. The stage is configured to be loaded with a sample S. The stage 101 of the microscope 10 can be a high-precision microscope stage.

The imaging assembly 12 may comprise a camera 121, an imaging light source 122, a focusing device 123, and a first shutter 124. The camera 121 is mounted on the microscope 10. In detail, the camera 121 is coupled to the microscope 10 through the subjective 103 of the microscope 10. The focusing device is coupled to the camera 121 and controlled to facilitate an autofocusing process during imaging of the sample S. The imaging light source 122, which provides an imaging light (as shown in the shaded area in FIG. 14A from imaging assembly 12 to the objective 102) through an imaging light path (as shown with the route indicated by the open arrows in the shaded area depicting the imaging light in FIG. 14A) to illuminate the sample S. The first shutter 124, along the imaging light path, is disposed between the image light source 122 and the microscope 10. The imaging light source 122 can be a tungsten-halogen lamp, an arc lamp, a metal halide lamp, a LED light, a laser, or multiple of them. The shuttering time of the first shutter may vary with the type of the imaging light source 121. Using an LED light source as an example, the shuttering time of the first shutter 124 is 20 microseconds.

If one would like to perform two color imaging, the shutter of the first color light is turned off and the shutter of the second color light is turned on by the first processing module 13. This may take another 40 microseconds. The camera 121 then takes another image with an exposure time of another 20 millisecond. The first processing module 13 then turns off the shutter of the second color light.

In this embodiment, please further refer to FIG. 14B, the illuminating assembly 11 comprises an illuminating light source 111, and a pattern illumination device 117 including a second shutter 112, a lens module 113 (such as the relay lens 113a and 113b, a quarter wave plate 113c), at least a pair of scanning mirrors 115 and a scan lens 116. Alternatively, DMD or SLM can be used as the pattern illumination device 117. The illuminating light source 111 provides an illuminating light (as shown in the open arrows from the illuminating assembly 11 to the objective 102 in FIG. 14A) through an illuminating light path to illuminate the sample S. The second shutter 112, along the illuminating light path, is disposed between the illuminating light source 111 and the microscope 10. The pair of scanning mirrors 115, along the illuminating light path, is disposed between the second shutter 112 and the microscope 10. The camera 121 may be a high-end scientific camera such as an sCMOS or an EMCCD camera with a high quantum efficiency, so that a short exposure time is possible. To get enough photons for image processing, the exposure time is, for example, 20 milliseconds.

The first processing module 13 is coupled to the microscope 10 and the imaging assembly 12. In detail, the first processing module 13 is coupled and therefore controls the camera 121, the imaging light source 122, the first shutter, the focusing device 123, and the stage 101 of the microscope 10, for imaging, focus maintenance, and changes of fields of view. The first processing module 13 can be a computer, a workstation, or a CPU of a computer, which is capable of executing a program designed for operating this system. The first processing module 13 then triggers the camera 121 to take the image of the sample S of a certain field of view (FOV). In addition, the camera 121 can be connected to the first processing module 13 through an USB port or a Camera Link thereon. The controlling and the image-processing procedures of this system will be discussed more detailed in the following paragraphs.

In this embodiment, the second processing module 14 is coupled to the illuminating assembly 11 and the first processing module 13. In detail, the second processing module 14 is coupled to and therefore controls the pattern illumination device 117, including the second shutter 112, and the pair of scanning mirrors, for illuminating the targeted points in the interested region determined by the first processing module 13. The second processing module may be a FPGA, an ASIC board, another CPU, or another computer. The controlling and the image-processing procedures of this system will be discussed more detailed in the following paragraphs.

In brief, the microscope-based system 1 is operated as below. The first processing module 13 controls the imaging assembly 12 such that the camera 121 acquires at least one image of the sample S of a first field of view. The image or images are then transmitted to the first processing module 13 and processed by the first processing module 13 automatically in real-time based on a predefined criterion, so as to determine an interested region in the image and so as to obtain a coordinate information regarding to the interested region. The image processing algorithm is developed independently beforehand using image processing techniques such as thresholding, erosion, filtering, or artificial intelligence trained semantic segmentation methods. Later, the coordinate information regarding to the interested region is transmitted to the second processing module 14. The second processing module 14 controls the illuminating assembly 12 to illuminate the interested region (or, namely, irradiating those targeted points in the interested region) of the sample S according to the received coordinate information regarding to the interested region. In addition, after the interested region is fully illuminated (or all the targeted points in the interested region are irradiated), the first processing module 13 controls the stage 101 of the microscope 10 to move to the next (i.e. the second) field of view which is subsequent to the first field of view. After moving to the subsequent field of view, the method further repeats imaging-image processing-illumination steps, until interested regions of all designated fields of view are illuminated.

Moreover, this disclosure also provides another embodiment which is a microscope-based method for image-guided microscopic illumination. The microscope-based method uses the microscope-based system described above and comprises the following steps (a) to (e): (a) triggering the camera 121 of the imaging assembly 12 by the first processing module 13 to acquire at least one image of the sample S of a first field of view, and the sample S is loaded on the stage 101 of the microscope 10; (b) automatically transmitting the image or images of the sample S to the first processing module 13; (c) based on a predefined criterion, performing image processing of the sample S automatically in real-time by the first processing module 13 to determine an interested region in the image and obtain a coordinate information regarding to the interested region; (d) automatically transmitting the coordinate information regarding to the interested region to the second processing module 14; (e) controlling an illumination assembly 11 by the second processing module 14 according to the received coordinate information to illuminate the interested region in the sample S. Besides, in this embodiment, after the interested region is fully illuminated, the method may further comprise a step of: controlling the stage 101 of the microscope 10 by the first processing module 13 to move to the next (i.e. the second) field of view which is subsequent to the first field of view.

The microscope-based system 1 used herein are substantially the same as that described above, and the details of the composition and variations of the compositing elements are omitted here.

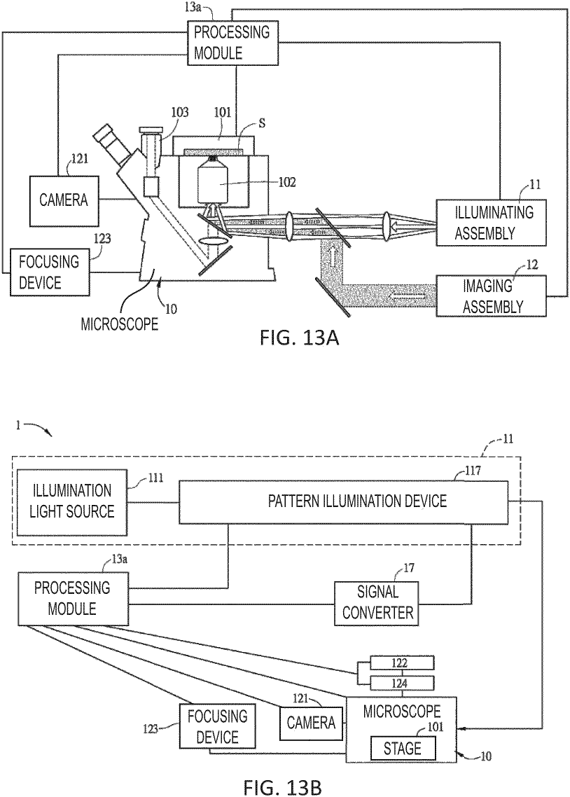

Moreover, as shown in FIGS. 13A and 13B, the light path of the illumination starts from the illumination light source 111. The second shutter 112 is needed for this illumination light source 111. To reach a high switching speed for the point illumination, a mechanical shutter may not be fast enough. One may use an acousto-optic modulator (AOM) or an electro optic modulator (EOM) to achieve the high speed. For example, an AOM can reach 25-nanosecond rise/fall time, enough for the method and system in this embodiment. After the second shutter 112, the beam size may be adjusted by a pair of relay lenses 113a and 113b. After the relay lenses 113a and 113b, the quarter wave plate 113c may facilitate to create circular polarization. The light then reaches the pairs of scanning mirrors (i.e. XY-scanning mirrors) 115 to direct the illumination light to the desired point one at a time. The light then passes a scan lens 116 and a tube lens (included in a microscope, not shown here) and the objective 102 of the microscope 10 to illuminate the targeted point of the sample S. An objective 102 with a high numerical aperture (NA) may be needed to have enough light intensity for photochemical reactions or photoconversion.

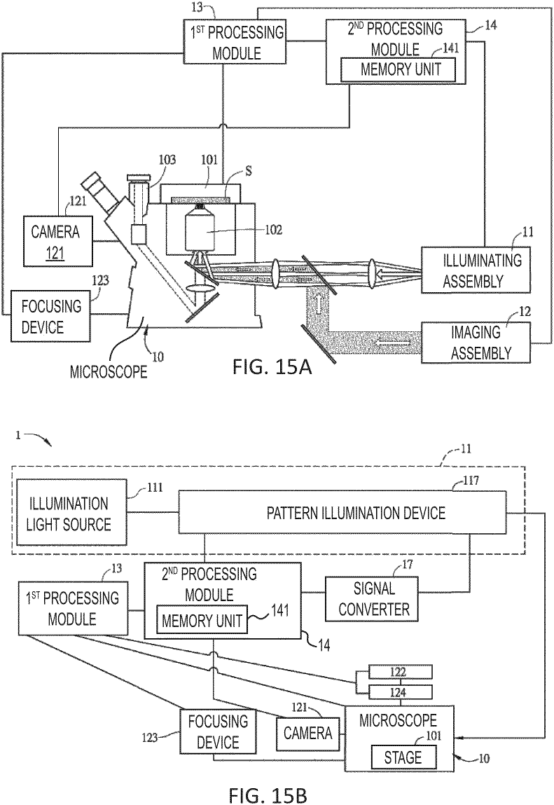

Also, this disclosure also provides another embodiment which is another microscope-based system for image-guided microscopic illumination. The microscope-based system for image-guided microscopic illumination is substantially the same as that is described above. Please refer to FIGS. 15A and 15B. In this embodiment, the microscope-based system 1 comprises a microscope 10, an illuminating assembly 11, an imaging assembly 12, a first processing module 13 and a second processing module 14. The microscope 10 comprises a stage 101, an objective 102 and a subjective 103, and the stage 10 is configured to be loaded with a sample S. Please further refer to both FIG. 15B, the illuminating assembly 11 comprises an illuminating light source 111, and a pattern illumination device 117 including a second shutter 112, at least one relay lens (such as the relay lens 113a and 113b), a quarter wave plate 113c, at least a pair of scanning mirrors 115 and a scan lens 116. Alternatively, DMD or SLM can also be used as the pattern illumination device 117. The imaging assembly 12 may comprise a camera 121, an imaging light source 122, a focusing device 123, and a first shutter 124. The camera 121 is mounted on the microscope 10.

The major difference between the systems described in the previous embodiment and here is that the first processing module 13 here is coupled to the stage 101 of the microscope 10 and the imaging light source 122 and the first shutter 124 of the imaging assembly 12. However, the second processing module 14 here comprises a memory unit 141 and is coupled to the camera 121, the illuminating assembly 11, and the first processing module 13. In other words, in this embodiment, the camera 121 is controlled by the second processing module 14 instead of the first processing module (i.e. the computer) 13. The camera 121 can be connected to the second processing module 14 through a Camera Link if a high speed of image data transfer and processing is required. The memory unit 141 can be a random access memory (RAM), flash ROM, or a hard drive, and the random access memory may be a dynamic random access memory (DRAM), a static random access Memory (SRAM), or a zero-capacitor random access memory (Z-RAM).

Hence, in the system 1 embodied here, it is operated as follows. The first processing module 13 controls the imaging assembly 12 and the second processing module controls 14 the camera 121 such that the camera 121 acquires at least one image of the sample S of a first field of view. The image or images are then automatically transmitted to the memory unit 141 to the second processing module 14. Image processing is then performed by the second processing module 14 automatically in real-time based on a predefined criterion, so as to determine an interested region in the image and so as to obtain a coordinate information regarding to the interested region. Later, the second processing module 14 controls the illuminating assembly 11 to illuminate the interested region of the sample S according to the received coordinate information regarding to the interested region.

Because composition, variation or connection relationship to other elements of each detail elements of the microscope-based system 1 can refer to the previous embodiments, they are not repeated here.

Also, this disclosure also provides still another embodiment which is another microscope-based method for image-guided microscopic illumination. The microscope-based method for image-guided microscopic illumination is substantially the same as that is described above. Please also refer to FIGS. 15A and 15B, the microscope-based method for image-guided microscopic illumination comprises the following steps through (a) to (d): (a) controlling the imaging assembly 12 by the first processing module 13 and triggering the camera 121 of the imaging assembly 12 by the second processing module 14 to acquire at least one image of the sample S of a first field of view, and the sample S is loaded on the stage 101 of the microscope 10; (b) automatically transmitting the image or images of the sample S to the memory unit 141 of the second processing module 14; (c) based on a predefined criterion, performing image processing of the sample S automatically in real-time by the second processing module 14 to determine an interested region in the image and to obtain a coordinate information regarding to the interested region; and (d) controlling the illuminating assembly 11 by the second processing module 14 to illuminate the interested region in the sample S according to the received coordinate information.

The wavelength of light for performing warhead activation or photoselective tagging and labeling ranges in some embodiments from about 200 nm to about 800 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, or from about 750 nm to about 800 nm. In some embodiments, the wavelength of light for performing photoselective tagging and labeling is short-wavelength UV light (e.g., 254 nm; 265-275 nm); long-UV light (e.g., 365 nm; 300-460 nm). The wavelength of light for performing warhead activation or photoselective tagging and labeling ranges in some embodiments from about 800 nm to about 2000 nm, e.g., from about 800 nm to about 900 nm, from about 900 nm to about 1000 nm, from about 1000 nm to about 1100 nm, from about 1100 nm to about 1200 nm, from about 1200 nm to about 1300 nm, from about 1300 nm to about 1400 nm, from about 1400 nm to about 1500 nm, from about 1500 nm to about 1600 nm, from about 1600 nm to about 1700 nm, from about 1700 nm to about 1800 nm, from about 1800 nm to about 1900 nm, or from about 1900 nm to about 2000 nm. In some embodiments, the wavelength of light for performing photoselective tagging and labeling is short-wavelength UV light (e.g., 254 nm; 265-275 nm); long-UV light (e.g., 365 nm; 300-460 nm). The wavelengths used for photoactivation of the warhead is different from the wavelengths used for imaging. In some embodiments, photoreactive warhead activation utilizes optical radiation (light) at from around 300-450 nm, 550 nm for single photon activation or >720 nm for multiphoton activation. The particular wavelength depends on the particular warhead. Cleavage can be driven by an enzyme or chemicals (such as sodium dithionite for cleaving azobenzene).

In some embodiments, a multivalent core (e.g., a core moiety) of a probe can be from around 70 Da to about 500 Da. A multivalent core can include or can be a single amino acid or a single nucleotide. In some embodiments, a core can be less than 1 nm in maximal width.

### Methods

Also disclosed herein are methods of photoselectively tagging and labeling biomolecules and analytical methods. The methods may be used to tag and/or label carbohydrates, lipids, nucleic acids, proteins, either alone or in combination. The methods may include the step of treating a biological sample with a bait molecule and a photoreactive and mildly cleavable probe and binding the bait molecule to a prey in the biological sample. In some embodiments, the probe includes a light-activated warhead and a tag and is bound to the bait molecule through a cleavable linker. Some embodiments include the step of illuminating the biological sample with an imaging lighting source of an image-guided microscope system. Some embodiments include the step of imaging the illuminated sample with a controllable camera. Some embodiments include the step of acquiring with the camera at least one image of subcellular morphology of the sample in a first field of view. Some embodiments include the step of processing the at least one image and determining a region of interest in the sample based on the processed image. Some embodiments include the step of obtaining coordinate information of the region of interest.

Some embodiments include the step of selectively illuminating with a crosslinking light the region of interest based on the obtained coordinate information to thereby doubly crosslink the probe and the bait. Some embodiments include the step of further comprising using the tag to generate a detectable label and labeling proteins proximal the prey with the detectable label. Some embodiments include the step of wherein the detectable label comprises a tyramine label. Some embodiments include the step of, wherein the biological sample comprises a plurality of cells. Some embodiments include the step of wherein the biological sample comprises a plurality of living cells. Some embodiments include the step of wherein the biological sample comprises cell extracts. Some embodiments include the step of wherein selectively illuminating comprises illuminating a region less than 300 nm, less than 200 nm, or less than 100 nm in diameter. Some embodiments include the step of further comprising removing at least the region of interest from the microscope stage.

Some embodiments include the step of further comprising subjecting the sample to mass spectrometry or sequencing analysis. Some embodiments include the wherein the tag comprises a biotin derivative, a click chemistry tag, a HaloTag, a SNAP-tag, a CLIP-tag, digoxigenin, or a peptide tag. Some embodiments include the wherein the click chemistry tag comprises an alkyne-based or azide-based moiety. Some embodiments include the wherein the cleavable linker is an azobenzene derivative, a Dde derivative, a DNA oligomer, a peptide, or a boronic acid ester. Some embodiments include the wherein the bait molecule comprises an antibody, protein A, protein G, protein L, a SNAP-tag, a CLIP-tag or a small molecule. Some embodiments include the wherein the light-activated warhead comprises an aryl azide, a diazirine, or a benzophenone.

Also described herein are photoselective tagging, labeling, and analyzing methods. methods. The methods may include the step of delivering a photoreactive and cleavable probe to a biological sample, wherein the probe comprises a cleavable linker, a light-activated warhead, and a tag and attached to a core of the probe. The methods may include the step of binding a bait molecule to a target biomolecule in the biological sample, wherein the bait molecule is conjugated to the probe. The methods may include the step of illuminating the biological sample from an imaging lighting source of an image-guided microscope system.

The methods may include the step of imaging the illuminated sample with a controllable camera. The methods may include the step of acquiring with the camera at least one image of subcellular morphology of the biological sample in a first field of view. The methods may include the step of processing the at least one image and determining a region of interest in the sample based on the processed image. The methods may include the step of obtaining coordinate information of the region of interest. The methods may include the step of selectively illuminating the region of interest with optical radiation to activate the light-activated warhead and attach the warhead to the target biomolecule or a target biomolecule neighbor such that the probe and target molecule are double-crosslinked. The methods may include the step of cleaving the cleavable linker of the probe. The methods may include the step of removing the cleaved and unbound probe.

Some embodiments include labeling a region less than 300 nm, less than 200 nm, or less than 100 nm in diameter. In some embodiments, the biological sample includes at least one, at least 100, at least 1000 or at least 10,000 live cells.

Some methods include contacting a biological sample having a target biomolecule with a probe as described herein, using optical radiation to spatially selectively photocrosslink the probe with a target biomolecule, cleaving the probe, washing unbound probe or cleaved probe away, labeling the biomolecule/probe complex with a label, and selectively proximity labeling biomolecule neighbor molecules.

### Kits

Also provided herein are kits and systems for practicing the methods described herein, e.g., for generating probes, and analyzing, tagging, and labeling biomolecules. Kits will typically include at least one photoreactive and cleavable probe as described herein or components thereof. In some embodiments, the at least one photoreactive and cleavable probe is configured to be mildly cleavable (e.g., bioorthogonally cleavable).

In addition, the kits will typically include instructional materials disclosing means for generating or modifying the one or more probes, such as e.g., attaching a bait moiety to the probe, applying the probe to a sample, conjugating the bait moiety to a prey molecule (in the sample), photocrosslinking the probe via the photoreactive warhead to a molecule of interest, photoreactively cleaving the cleavable linker via the cleavable linker bond, removing (washing away) non-photoreactive probe,

The kits may also include additional components to facilitate the particular application for which the kit is designed. Thus, for example, where a kit contains one or more photoreactive and cleavable probe for tagging and labelling biomolecules, the kit can additionally contain one or more cleavage molecule (e.g., a chemical, an endonuclease, a protease). The kit can additionally contain one or more bait molecules, such as any of those described herein (e.g., an antibody, a functional protein (e.g., protein A, protein G, a protein drug, etc.), a self-labeling protein (e.g., a CLIP-tag, a Halo-Tag, a SNAP-tag), a small molecule or drug.

The kit can additionally contain means of detecting the sample and/or detecting the label (e.g., enzyme substrates for enzymatic labels, filter sets to detect fluorescent labels, enzymes or associated detection reagents, including reagents for performing catalyzed reporter deposition (CARD) or signal amplification (e.g., avidin, Neutravidin, streptavidin, HRP, tyramide, hydrogen peroxide, etc.). The kits may additionally include wash solutions, such as blocking agents, detergents, salts (e.g., sodium chloride, potassium chloride, phosphate buffer saline (PBS)) for one or more steps (e.g., after sample fixation, after probe cleavage, etc.). A kit may include variations of wash solutions, such as concentrates of wash buffers configured to be diluted before use or components to use for making one or more wash solutions) and other reagents routinely used for the practice of a particular method. A kit may include fixatives and other sample preparation materials (e.g., ethanol, methanol, formalin, paraffin, etc.)

The kits can optionally include instructional materials teaching the use of the probes, cleavage molecules, addition of a bait molecule to a probe, and wash solution and the like.

### Experimental and Methods

Example 1 Demonstration of successful localized photoselective tagging of nucleolin using azo probe. FIG. 12A shows a schematic of photoselective tagging of nucleolin. Nucleolin is a protein found in the nucleolus of eukaryotic cells and involved in the synthesis of ribosomes. Azo-probe 1 was conjugated to a secondary antibody by using BCN-NHS (CAS# 1516551-46-4) as additional linker between Azo-probe 1 and secondary antibody. A sample of U2OS cells was grown on a glass-bottom chamber slide and fixed with 2.4% PFA. The antibody conjugated with Azo-probe 1 was applied to the sample stained with anti-nucleolin antibody. The sample was exposed to 780 nm two-photon irradiation (200mW, 200 µs/pixel) to photocrosslink the light activated warhead to the antibody and subsequently incubated with 1 M sodium dithionite at room temperature for over 16 h to remove non-crosslinked probes. Neutravidin conjugated to Alexa Fluor 647 dye was added and the sample assayed for the Alexa Fluor 647. Alexa Fluor 647 is a bright, far-red-fluorescent dye with excitation ideally suited for the 594 nm or 633 nm laser lines. Results are shown in the top panel of FIG. 12B. A close-up view is shown in the top right side of FIG. 12B. The characteristic nucleoli shape is observed. A side view is shown in the bottom right of FIG. 12B. The bottom of FIG. 12B shows a control region treated the same as in the top panel except that the sample shown in the bottom panel was not exposed to photoactivating light. No significant staining was observed.

### Example 2

### Preparation of BCN-antibody

1. For 100 µl of reaction, prepare 70 µl antibody (1.2-1.5 µg/µl) of solution. 2. Add 10 µl of 1 M sodium bicarbonate (or 1 M borate buffer, final 50-100 mM) and BCN-NHS (Sigma-Aldrich #744867, final concentration: 200 µM). Adjust the final volume to 100 µlwith ddH2O. 3. Mix gently by inverting the tube a few times and mildly spin down. 4. Incubate on shaker/mixer for 1 hour at room temperature. Avoid from light if needed. 5. Stop the reaction by adding 10 µl of 1 M glycine and react for another 30-60 minutes at room temperature. 6. Remove non-conjugated small molecules by resin filtration using desalting column.

Preparation of Probe 3-antibody conjugate:7. Mix 0.5-1 µg/µl antibody with probe 3 (final concentration: 100 µM), react overnight at 4° C. 8. Remove non-conjugated small molecules by resin filtration using desalting column.

Photoselective labeling: 9. Treat nucleolin-stained cells with Probe 3-antibody and DRAQ5 (nucleus marker) in PBS solution supplement with 0.1% triton for 60 min. 10. Wash the sample with PBS solution supplement with 0.1% triton and fix the sample with 2.4% PFA. 11. Define desired area and label the Probe 3-antibody stained nucleolin within the selected area with 160-200 mW pulsed laser at 780 nm.12. Wash the labeled samples with PBS and incubate with 1 M sodium dithionite overnight at RT.13. Check the labeling by staining with NeutrAvidin-Dy550 conjugates (1:200).

### Example 3

### Preparation of Probe IV N-TEV

Pre-conjugated peptides N-TEV were dissolved in DMSO/Water (1/1) to 1 mM. N-Succinimidyl 4-Benzoylbenzoate (TCI # S0863) were dissolved in pure anhydrous DMSO to 2 mM. 10 µL of N-TEV stock solution were mixed with 10 µL of N-Succinimidyl 4-Benzoylbenzoate stock solution, 10 µL of 1 M sodium borate buffer (pH=8.5), 70 µL of DMSO/Water (1/1) and react for 2 h at room temperature. The reaction was quenched by adding 10 µL of 1 M glycine solution and validated with MALDI-MS.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A photoreactive and cleavable probe, comprising:
a multivalent core comprising a plurality of attachment sites;
a tag bound to one of the attachment sites, wherein the tag is configured to conjugate to a label;
a cleavable linker bound to a second of the attachment sites and configured to link to a bait molecule, wherein the cleavable linker comprises a cleavable linker bond other than a disulfide bond; and
a light-activated warhead bound to a third of the attachment sites.

2. The photoreactive and cleavable probe of claim 1, wherein the probe is bioorthogonally cleavable.

3. The photoreactive and cleavable probe of claim 1, wherein the tag comprises a biotin derivative, a CLIP-tag, a click chemistry tag, digoxigenin, a HaloTag, a peptide tag, or a SNAP-tag.

4. The photoreactive and cleavable probe of claim 3, wherein the biotin derivative includes the moiety of 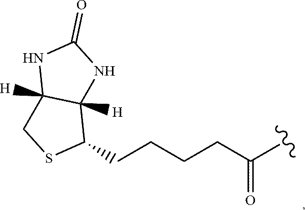 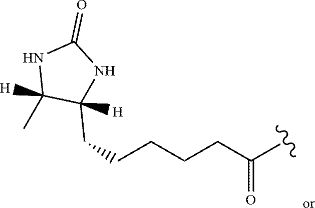 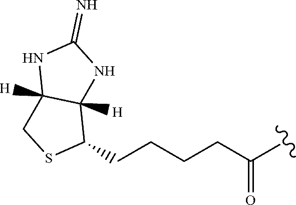
.

5. The photoreactive and cleavable probe of claim 3, wherein the click chemistry tag comprises an alkyne-based or azide-based moiety.

6. The photoreactive and cleavable probe of claim 3, wherein the click chemistry tag includes the moiety of 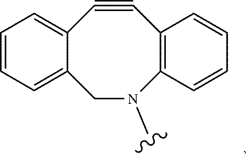 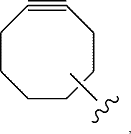 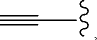 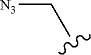
or 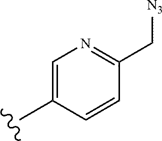
.

7. The photoreactive and cleavable probe of claim 1, wherein the cleavable linker comprises an azobenzene derivative, a boronic acid ester, a Dde derivative, a DNA oligomer, or a specifically cleavable peptide.

8. The photoreactive and cleavable probe of claim 7, wherein the azobenzene derivative includes the moiety of 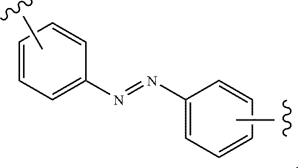
.

9. The photoreactive and cleavable probe of claim 7, wherein the Dde derivative includes the moiety of 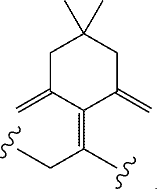
.

10. The photoreactive and cleavable probe of claim 1, wherein the bait molecule comprises an antibody, a CLIP-tag, a HaloTag, protein A, protein G, protein L, an RNA molecule, a small molecule, or a SNAP-tag.

11. The photoreactive and cleavable probe of claim 1, wherein the light-activated warhead comprises an aryl azide, a benzophenone, or a diazirine.

12. The photoreactive and cleavable probe of claim 11, wherein the aryl azide comprises the moiety of 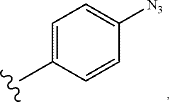 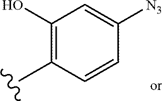 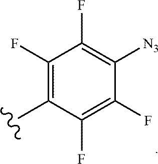
.

13. The photoreactive and cleavable probe of claim 11, wherein the diazirine includes the moiety of 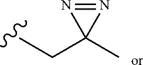 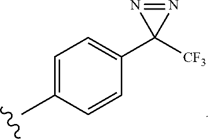
.

14. The photoreactive and cleavable probe of claim 11, wherein the benzophenone includes the moiety of 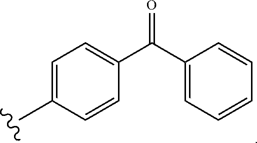
.

15. The photoreactive and cleavable probe of claim 1, wherein the light-activated warhead comprises a nucleobase-specific 3-cyanovinylcarbazole nucleoside (CNVK), including the moiety of 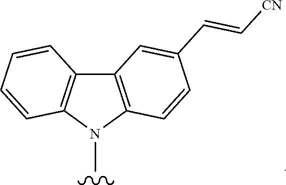
.

16. The photoreactive and cleavable probe of claim 1, wherein the light-activated warhead comprises a nucleobase-specific psoralen, including the moiety of 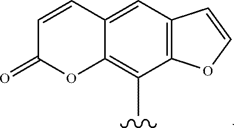
.

17. The photoreactive and cleavable probe any claim 1, wherein the phenoxyl radical trapper comprises a light-activated warhead, including the moiety 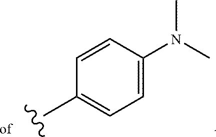
.

18. The photoreactive and cleavable probe of claim 1, wherein the cleavable linker comprises azobenzene, boronic ester, a Dde moiety, a DNA oligomer, or a cleavable peptide.

19. The photoreactive and cleavable probe of claim 1, wherein the cleavable linker comprises a human rhinovirus 3C (HRV 3C) protease recognition sequence or a tobacco etch virus (TEV) protease recognition sequence.

20. The photoreactive and cleavable probe of claim 1, wherein the multivalent core includes the moiety of formula (I): 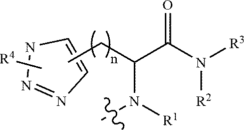
wherein n is 1, 2, 3, 4, 5, or 6;
R¹ and R² each independently are hydrogen, substituted
alkyl, substituted alkenyl, substituted alkynyl, substituted carbocyclyl, substituted heterocyclyl, substituted aryl, substituted heteroaryl, or a nitrogen protecting group; and
one of R³ and R⁴ is - (CH₂)ₓ(OCH₂CH₂)_{y}(CH2)_{z}NR⁵R⁶, and the other is an attachment site,
wherein x is 1, 2, 3, 4, 5, or 6;
y is 1, 2, 3, 4, 5, or 6;
z is 0, 1, 2, 3, 4, 5, or 6; and
one of R⁵ and R⁶ is an attachment site, and the other is
hydrogen, substituted alkyl, substituted alkenyl, substituted alkynyl, substituted carbocyclyl, substituted heterocyclyl, substituted aryl, substituted heteroaryl, or a nitrogen protecting group.

21. The photoreactive and cleavable probe of claim 20, wherein the multivalent core comprises the moiety of formula (I-1) or (I-2): 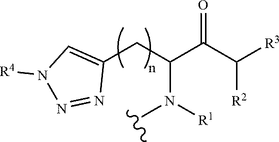 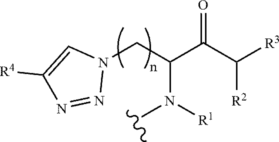
.

22. The photoreactive and cleavable probe of claim 21, wherein the multivalent core comprises the moiety of: 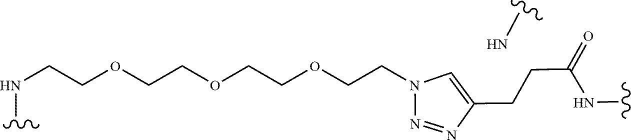 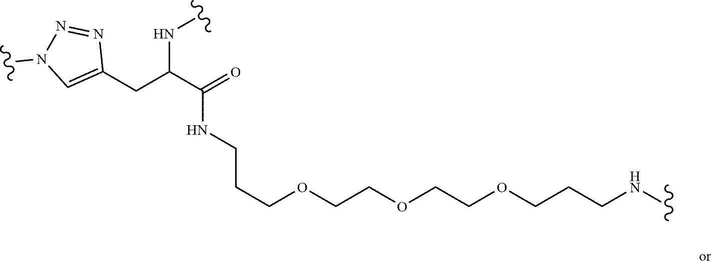 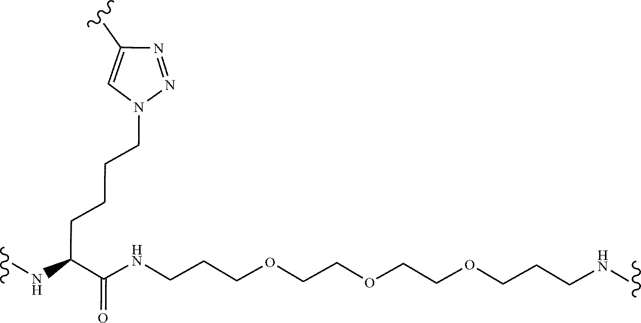
.

23. The photoreactive and cleavable probe of claim 1 , which comprises the following structure: 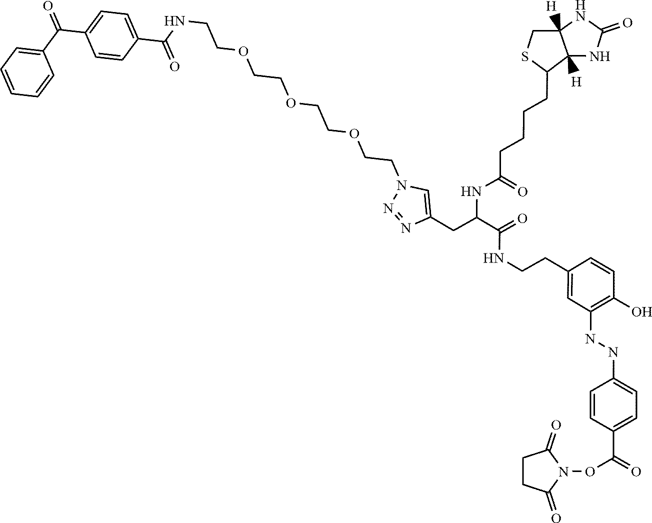 [SMILES: O=C(CCCCC1SC[C@@H]2NC(=O)N[C@H]12)NC(Cc1cn(CCOCCOCCOCCNC(=O)c2ccc(C(=O)c3ccccc3)cc2)nn1)C(=O)NCCc1ccc(O)c(NNc2ccc(C(=O)ON3C(=O)CCC3=O)cc2)c1] 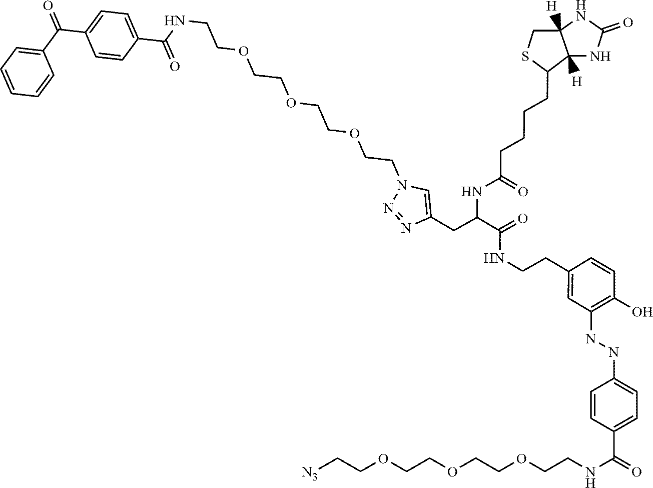 [SMILES: [N-]=[N+]=NCCOCCOCCOCCNC(=O)c1ccc(NNc2cc(CCNC(=O)C(Cc3cn(CCOCCOCCOCCNC(=O)c4ccc(C(=O)c5ccccc5)cc4)nn3)NC(=O)CCCCC3SC[C@@H]4NC(=O)N[C@H]34)ccc2O)cc1] 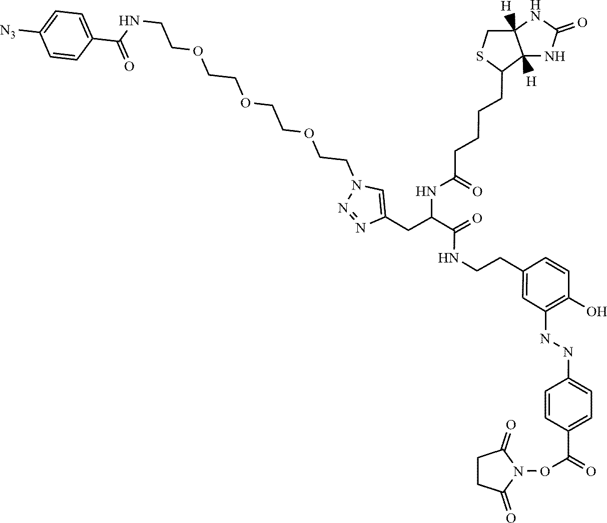 [SMILES: [N-]=[N+]=Nc1ccc(C(=O)NCCOCCOCCOCCn2cc(CC(NC(=O)CCCCC3SC[C@@H]4NC(=O)N[C@H]34)C(=O)NCCc3ccc(O)c(NNc4ccc(C(=O)ON5C(=O)CCC5=O)cc4)c3)nn2)cc1] 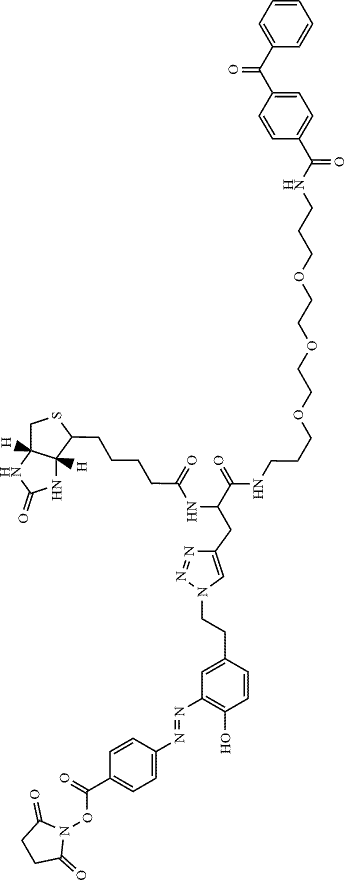 [SMILES: O=C(CCCCC1SC[C@H]2NC(=O)N[C@@H]12)NC(Cc1cn(CCc2ccc(O)c(/N=N/c3ccc(C(=O)ON4C(=O)CCC4=O)cc3)c2)nn1)C(=O)NCCCOCCOCCOCCCNC(=O)c1ccc(C(=O)c2ccccc2)cc1]
, 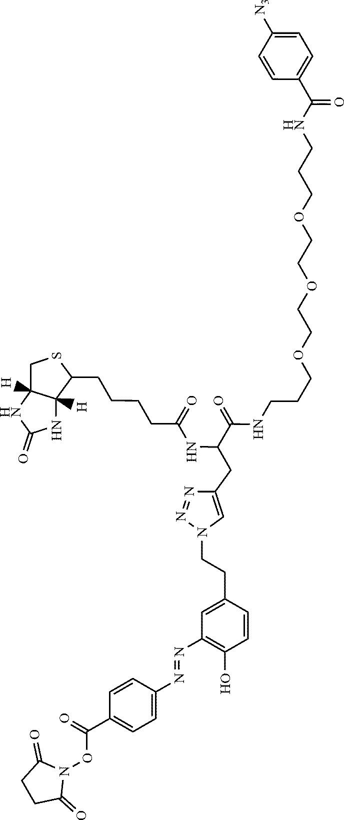 [SMILES: [N-]=[N+]=Nc1ccc(C(=O)NCCCOCCOCCOCCCNC(=O)C(Cc2cn(CCc3ccc(O)c(/N=N/c4ccc(C(=O)ON5C(=O)CCC5=O)cc4)c3)nn2)NC(=O)CCCCC2SC[C@H]3NC(=O)N[C@@H]23)cc1] 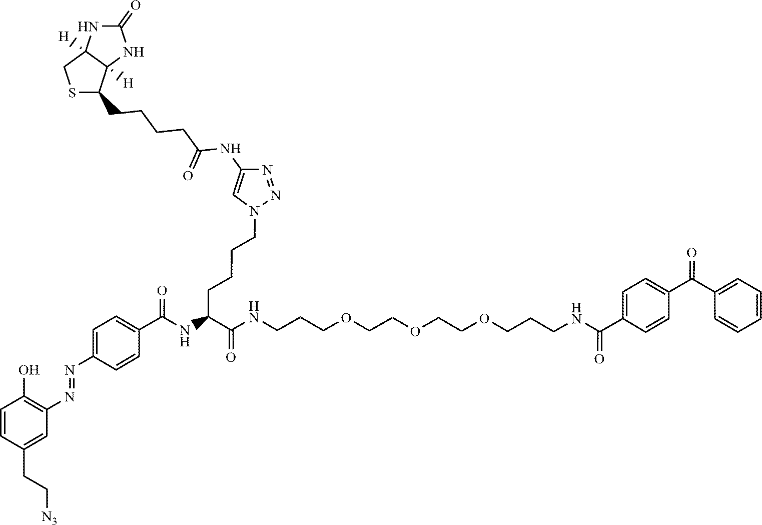 [SMILES: [N-]=[N+]=NCCc1ccc(O)c(/N=N/c2ccc(C(=O)N[C@@H](CCCCn3cc(NC(=O)CCCC[C@H]4SC[C@H]5NC(=O)N[C@H]54)nn3)C(=O)NCCCOCCOCCOCCCNC(=O)c3ccc(C(=O)c4ccccc4)cc3)cc2)c1]
, or 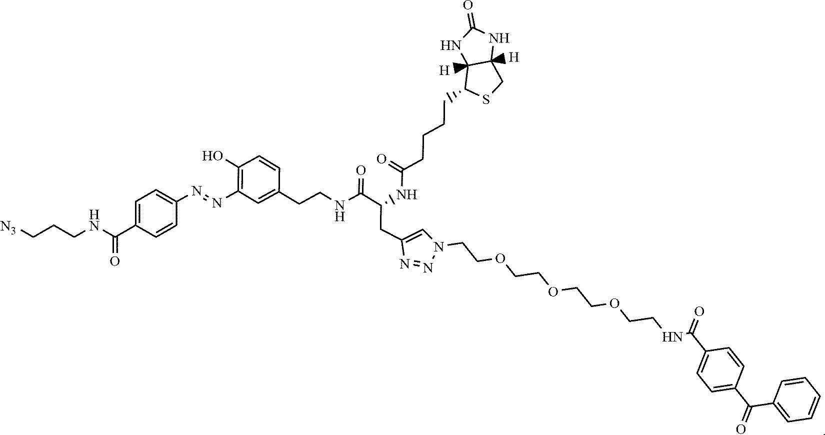
.

24. The photoreactive and cleavable probe of claim 23, wherein probes 2 and 6 further include an additional linker molecule configured for linking the probes 2 and 6, respectively, to the bait molecule.

25. The photoreactive and cleavable probe of claim 1, further comprising a flexible linker.

26. The photoreactive and cleavable probe of claim 1, further comprising a flexible linker comprising polyethylene glycol (PEG) or an (GGGGS)n oligomer (SEQ ID NO: 16).

27. A method for photoactivated labeling comprising:
delivering the photoreactive and cleavable probe as claimed in claim 1 to a biological sample, wherein the photoreactive and cleavable probe is linked to the bait molecule;
conjugating the bait molecule to a target biomolecule in the biological sample to crosslink the probe and target biomolecule;
delivering optical radiation to activate the light-activated warhead of the
photoreactive and cleavable probe and attach the warhead to the target biomolecule or a target biomolecule neighbor such that the probe and target biomolecule are double-crosslinked;
cleaving the cleavable linker of the probe such that probe that is not double-crosslinked to the target biomolecule or a target biomolecule neighbor is cleaved; and
removing the cleaved and unbound probe.

28. An analytical method comprising:
delivering a photoreactive and cleavable probe to a biological sample, wherein the probe comprises a cleavable linker, a light-activated warhead, and a tag and attached to a core of the probe;
conjugating the bait molecule to a target biomolecule in the biological sample to crosslink the probe and target biomolecule;
illuminating the biological sample from an imaging lighting source of an image-guided microscope system;
imaging the illuminated sample with a controllable camera;
acquiring with the camera at least one image of subcellular morphology of the biological sample in a first field of view;
processing the at least one image and determining a region of interest in the sample based on the processed image;
obtaining coordinate information of the region of interest;
selectively illuminating the region of interest with optical radiation to activate the light-activated warhead and attach the warhead to the target biomolecule or a target biomolecule neighbor such that the probe and target molecule are double-crosslinked;
cleaving the cleavable linker of the probe such that probe that is not double-crosslinked to the target biomolecule or a target biomolecule neighbor is cleaved; and
removing the cleaved and unbound probe.

29. The method of claim 27, wherein cleaving the cleavable linker comprises performing a bioorthogonal cleavage reaction.

30. The method of claim 27, wherein the cleavable linker comprises a cleavable linker bond and the step of cleaving the cleavable linker comprises cleaving a bond other than a disulfide bond.

31. The method of 30 claim 27, further comprising conjugating a detectable label with the tag of the probe and detectably proximity labeling neighbors proximal the target biomolecule by detectable label activity.

32. The method of claim 31, wherein detectably proximity labeling comprises photoselective proximity labeling a region less than 300 nm, less than 200 nm, or less than 100 nm in diameter.

33. The method of claim 31, wherein the detectable label comprises a catalytic label.

34. The method of claim 27, wherein the biological sample comprises a plurality of cells.

35. The method of claim 27, wherein the biological sample comprises at least one, at least 100, at least 1000 or at least 10,000 live or fixed cells.

36. The method of claim 27, wherein the biological sample comprises fixed cells, tissues or cell or tissue extracts.

37. The method of claim 27, wherein selectively illuminating comprises illuminating a zone defined by point spread function.

38. The method of claim 27, wherein the biological sample is disposed on a microscope stage, the method further comprising removing at least a portion of the biological sample region of interest from the stage.

39. The method of claim 27, further comprising subjecting the sample to mass spectrometry analysis or sequencing analysis.

40. The method of claim 27, wherein the tag comprises a biotin derivative, a CLIP-tag, a click chemistry tag, digoxigenin, a HaloTag, a peptide tag, or a SNAP-tag.

41. The method of claim 27, wherein the click chemistry tag comprises an alkyne-based or azide-based moiety.

42. The method of claim 27, wherein the cleavable linker comprises an azobenzene derivative, a boronic acid ester, a Dde derivative, a DNA oligomer, or a peptide.

43. The method of claim 27, wherein the bait molecule comprises an antibody, a CLIP-tag, a HaloTag, protein A, protein G, protein L, a small molecule, or a SNAP-tag.

44. The method of claim 27, wherein the light-activated warhead comprises an aryl azide, a benzophenone, or a diazirine.

45. A photoreactive and cleavable probe comprising:
a multivalent core comprising a plurality of attachment sites;
a tag bound to one of the attachment sites, wherein the tag is configured to conjugate to a label;
a cleavable linker bound to a second of the attachment sites and configured to link to a bait molecule, wherein the cleavable linker comprises a peptide sequence;
a light-activated warhead bound to a third of the attachment sites,
wherein the multivalent core comprises the moiety of formula (II) or (III): 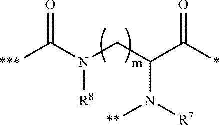 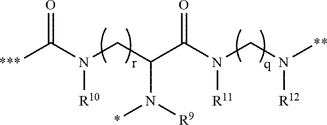
wherein m, r and q each independently are 1, 2, 3, 4, 5, or 6;
wherein * comprises an attachment site of one of the plurality of attachment sites for the cleavable linker, wherein ** comprises a different attachment site of the plurality of attachment sites for one of either the tag or the photoreactive warhead; wherein *** comprises a different attachment site of the plurality of attachment sites for either the photoreactive warhead or the tag, respectively, and R7, R8, R9, R10, R11, and R12 each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

46. The photoreactive and cleavable probe of claim 45, wherein ** comprises the attachment site for the tag, and *** comprises the attachment site for the photoreactive warhead.

47. The photoreactive and cleavable probe of claim 45, wherein the peptide sequence comprises a protease recognition sequence.

48. The photoreactive and cleavable probe of claim 45, wherein the peptide sequence comprises a human rhinovirus 3C (HRV 3C) protease recognition sequence, a tobacco etch virus (TEV) protease recognition sequence, or a thrombin recognition sequence.

49. The photoreactive and cleavable probe of claim 45, wherein the cleavable linker further comprises a conjugatable amino acid configured to conjugate to a bait molecule.

50. The photoreactive and cleavable probe of claim 45, wherein the cleavable linker further comprises a cysteine or clickable amino acid amino acid.

51. The photoreactive and cleavable probe of claim 45, wherein the cleavable linker comprises a clickable amino acid with an azido or alkyne moiety.

52. A kit for labeling biomolecules comprising:
the photoreactive and cleavable probe of claim 1 in a first container; and
an instructional material.

53. A kit for labeling biomolecules comprising:
a multivalent core moiety in a first container, wherein the multivalent core comprises a plurality of attachment sites;
a tag configured to conjugate to a label and bound to or configured to bind to the multivalent core moiety;
a cleavable linker comprising a cleavable linker bond other than a disulfide bond, wherein the cleavable linker is linked to or configured to link to a bait molecule;
a light-activated warhead bound to or configured to bind to a third attachment site on the multivalent core moiety; and
an instructional material.

54. The kit of claim 53 wherein at least one of the tag, the cleavable linker, and the light-activated warhead are separate from the multivalent core.

55. The kit of claim 52, further comprising a linker cleavage molecule.

56. The kit of claim 55, wherein the linker cleavage molecule comprises an endonuclease or a site-specific protease.

57. The kit of claim 55 wherein the linker cleavage molecule comprises human rhinovirus 3C (HRV 3C) protease or tobacco etch virus (TEV) protease.

58. The kit of claim 55, wherein the linker cleavage molecule comprises factor X enteropeptidase or thrombin.

59. The kit of above claim 52, further comprising one or more of: an antioxidant, a buffering agent, a detergent, a nuclease inhibitor, a stabilizing agent, and a wash agent.

60. The kit of claim 52, further comprising a bait molecule.

61. The kit of claim 52, further comprising a detectable label.

62. The kit of claim 52, further comprising a label that specifically conjugates with biotin.

63. The kit of claim 52, further comprising a fixative solution.

64. The kit of claim 53 wherein the multivalent core, the tag, the cleavable linker, and the light-activated warhead are present in the same molecule.
